(19) United States
(12) Patent Application Publication
(10) Pub. No.: US 2005/0084547 A1
(43) Pub. Date: Apr. 21, 2005

(54) Natural product based apoptosis inducers

(75) Inventor: Ven Subbiah, Greenville, NC (US)

Correspondence Address: DANIELS DANIELS & VERDONIK, P.A., SUITE 200 GENERATION PLAZA, 1822 N.C. HIGHWAY 54 EAST, DURHAM, NC, 27713, US

(73) Assignee: PhytoMyco Research Corporation, Greenville, NC (US)

(21) Appl. No.: 10/933,717

(22) Filed: Sep. 3, 2004

Related U.S. Application Data
(60) Provisional application No. 60/502,564, filed on Sep. 12, 2003.

(51) Int. Cl. A61K 35/78; A61K 31/704

(52) U.S. Cl. 424/740; 514/26

(57) ABSTRACT

Pharmaceutical compositions are made from extracts obtained from ethnobotanical plants for inducing apoptosis in selected cells. Therapeutically effective amounts of the composition are administered to a mammal. Assays are used to determine the efficacy of such extracts in inducing apoptosis.

### CROSS REFERENCE TO RELATED APPLICATIONS

This application is related to and claims priority to U.S. Provisional Application Ser. No. 60/502,564, filed Sep. 12, 2003, the disclosure of which is incorporated in its entirety by reference herein.

### FIELD OF THE INVENTION

This invention relates to anticancer drugs and to pharmaceutical compositions and methods for induction of apoptosis in diseased cells in human and animal patients and to methods useful to identify such compounds. In particular, this invention relates to the use of compositions comprising plant extracts and containing sclareol, sclareolide, sclareo-like, and sclareolide-like compounds for induction of apoptosis in diseased cells, particularly in cancer cells. The present invention also relates to methods for screening a mixture of components of an extract of a plant or other natural source using a fluorescent-based ligand-receptor interaction assay technique to identify a compound or a mixture of compounds that exhibits activity in the induction of apoptosis in diseased cells, particularly in cancer cells.

### BACKGROUND OF THE INVENTION

Apoptosis is a programmed cell death, herein referred to as PCD, which comprises a series of programmed intracellular events that lead to death of a cell. Apoptosis is a programmed individual cell death that occurs normally in development, during aging and in various pathologic conditions. It is a highly organized physiological mechanism to destroy, for example, injured or abnormal cells. Apoptosis is normally an active process requiring metabolic activity by a dying cell, and is often characterized by cleavage of DNA in the cell nucleus into DNA fragments that give a laddering pattern on gels. Cells that die by apoptosis do not usually elicit an inflammatory response associated with necrosis. Cancerous cells are usually unable to experience normal cell transduction or an apoptosis-driven natural cell death process.

Apoptosis and necrosis differ in both biochemical and morphological changes that occur in a cell. Apoptotic cells are characterized morphologically by compaction of the nuclear chromatin, shrinkage of the cytoplasm, and production of membrane-bound apoptotic bodies. Apoptosis is distinguished biochemically by fragmentation of the genome and cleavage or degradation of several cellular proteins. Apoptotic cells are usually eliminated by phagocytosis. The breakdown of the nucleus of a cell during the process of apoptosis involves collapse and fragmentation of the chromatin, degradation of the nuclear envelope, and nuclear blebbing. This results in the formation of micronuclei.

Cytotoxic drugs or anticancer agents can act on a cell in a number of ways to induce cell death. In one manner, a cell can die or be killed by an injurious or cytotoxic agent that causes injury to the cell. When a cell is killed in this manner, it can undergo a series of changes, for example, the cell and organelles such as mitochondria can swell by osmotic mechanisms related in part to damage of plasma membrane which modifies or eliminates control the passage of ions and water into and out of the cell. Cell contents can leak out and inflammation of surrounding tissue can occur.

A cell that undergoes apoptosis or programmed cell death can shrink; its mitochondria can break down with the release of cytochrome c; it can develop bubble-like blebs on its surface; chromatin (DNA and protein) in its nucleus degrades and breaks into small, membrane-wrapped, fragments; phosphatidylserine, normally hidden within its plasma membrane is exposed on the surface and becomes bound by receptors on phagocytic cells such as macrophages and dendritic cells which then engulf the cell fragments. These phagocytic cells secrete cytokines that inhibit inflammation.

In a healthy cell, the outer membranes of its mitochondria express the protein Bcl-2 on their surface. Bcl-2 is bound to a molecule of the protein Apaf-1. Internal damage to the cell (e.g., from reactive oxygen species) causes Bcl-2 to release Apaf-1 and a related protein, Bax, to penetrate mitochondrial membranes, causing cytochrome c to leak out. The released cytochrome c and Apaf-1 bind to molecules of caspase 9. The resulting complex of cytochrome c, Apaf-1, caspase 9 (and ATP) is known as an apoptosome. These aggregate in the cytosol. Caspase 9 is one of a family of over a dozen caspases which are all proteases which cleave proteins, mostly each other, at aspartic acid (Asp) residues. Caspase 9 cleaves and, in so doing, activates other caspases. The sequential activation of one caspase by another creates an expanding cascade of proteolytic activity which leads to digestion of structural proteins in the cytoplasm, degradation of chromosomal DNA, and phagocytosis of the cell.

Apoptotic cells exhibit characteristic morphological features and molecular expression. Apart from physiological stimuli, there are exogenous factors which can contribute to induction of apoptosis. The induction of apoptosis in tumor cells is considered very useful in the management and therapy as well as in the prevention of cancer.

Caspases are programmed cell death gene products that, when activated cause cells to undergo apoptosis. Drug candidates that drive cancer cells into apoptosis independent of the tumor suppressor gene p53 are highly desirable. P53-independent anticancer drugs have great potential for cancer therapy, because a large percentage, 50% or more, of all cancers have mutations of p53. This causes cancer cells to develop resistance to treatment with conventional chemotherapeutic agents. Compounds that specifically activate caspases in multidrug resistant cells have strong potentials to become useful therapeutic agents.

Caspases are vital to programmed cell death. The role of caspase in diacetyldianhydrogalactitol (DADAG)-induced apoptosis in human leukemia HL-60 cells has been identified—see Yang et al., Acta Pharmacol Sin. (2002) May; 23(5):461. An MTT assay was used to measure cell proliferation with trypan blue and propidium iodide to detect dead cells. Apoptosis was observed by microscopy, flow cytometry, and DNA fragmentation assay. A combined western blot and ApoAlert CPP32 colorimetric assay kit allowed the caspase-3 activity to be measured by substrate cleavage. Results revealed that DADAG induced apoptosis in the HL-60 cells by polymerase (PARP), lamin B, and DFF45.

Preferred apoptotic inducers should not be cytotoxic to normal tissues and to the immune cell system.

Cancer can result from a perturbation in one or more cellular pathways that lead to normal cell proliferation, differentiation and death. Currently available treatment of cancer can consist of surgical removal of tumor tissue such as surgical de-bulking of a solid tumor, cytotoxic radiation of a cancerous lesion, systemic or local administration of a cytotoxic drug, and combinations thereof.

Cytotoxic cancer chemotherapeutic agents can provide temporary relief from symptoms associated with tumor growth, prolongation of life of a patient that has a cancer, and occasionally, cure of the cancer. Cancer chemotherapy can target cell proliferation.

Programmed cell death (PCD) pathways are important mediators of cancer. PCD includes apoptosis, with distinct morphological changes including chromatin condensation. Mutations in a cell that lead to proliferation can also induce apoptosis, in part because the cell is programmed to maintain a delicate balance of growth and death, and apoptotic pathways in this balance are often mutated during carcinogenesis. For example, many types of cancer have a mutation in the p53 gene, whose product controls the cell's progression to live or to die, often as a function of the state of the cell. In addition to the pathways that cause cells to proliferate, pathways that control apoptosis have an important role in normal cell function.

A successful anticancer drug should kill or incapacitate cancer cells without causing damage, particularly excessive damage, to normal and non-cancerous cells. For example, anti-estrogens such as tamoxifen can be used as chemotherapeutic agents against breast cancers that are dependent on estrogens for growth. However, anti-proliferative drugs such as tamoxifen are specific only for types of cancer that rely on external growth signals. Anti-proliferative drugs such as methotrexate, which inhibits purine synthesis, can succeed because of the difference induced in proliferation rates between cancerous and normal cells. Such anti-proliferative drugs as methotrexate can still be quite toxic to normal cells.

Dysfunction of the apoptotic pathway can lead to cancer, and modulating apoptosis can be useful in the management, therapy, and/or prevention of cancer. Apoptosis can be modulated by compositions of this invention. Modulation can comprise initiation of the process of apoptosis in a cancerous cell and/or acceleration of the process of apoptosis in a cancerous cell. It is an advantage that the compositions of this invention can modulate the process of apoptosis in a cancerous cell.

A more promising approach to the treatment of cancer is to induce cell death specifically in cancer cells by apoptosis. Apoptosis induction is a possible mechanism of action for some current anti-tumor treatments and agents, including ionizing radiation, alkylating agents such as cisplatin and 1,3-bis(2-chloroethyl)-1-nitrosourea (BCNU), the topoisomerase inhibitor etoposide, cytokine tumor necrosis factor (TNF), and Taxol, although a number of drugs kill cancer cells by alternate mechanisms.

In the last several years, apoptosis has been a target for mechanism-based drug discovery. Synthetic modification of known drugs in an attempt to increase therapeutic indices and efficacies is an important aspect of research toward cancer treatment. However, a vast amount of synthetic work has contributed only relatively small improvements over the prototype drugs in many cases. There is a continued need for new prototype drugs as new templates to use in the design of potential chemotherapeutic agents. Significantly, natural products are providing such templates. It is an advantage that compositions of this invention can provide new natural product templates as prototype drugs for use in the treatment of cancer, for example, as agents that are cytotoxic to cancer cells, as agents that induce apoptosis, as agents that modulate apoptosis, and the like.

Cells in a multi-cellular organism require a signal to stay alive. When the signal is not present, which can be referred to as trophic factors, the cells initiate a suicide program. Cancer cells have taken advantage of these activators to remain alive. Trophic factor receptors are located on the surface of the plasma membrane. When activated the receptor begins a cascade of protein interaction and release leading to cell death. Cellular cascade of activated trophic factor receptor initiating apoptosis. The cell has not received the trophic factor that will inhibit apoptosis. Bad, a soluble pro-apoptotic protein, binds to the anti-apoptotic proteins Bcl-2 and Bcl-xl, which are inserted into the mitochondrial membrane. Bad binding prevents the anti-apoptoic proteins from interacting with Bax, a membrane-bound pro-apoptotic protein. Consequently, Bax forms homo-oligomeric channels in the membranes that mediate ion flux. Through an as-yet-unknown mechanism, this leads to the release of cytochrome c from the space between the inner and outer mitochondria membrane. Cytochrome c then binds to the adapter protein Apaf-1, which in turn promotes a caspase cascade leading to cell death.

### SUMMARY OF THE INVENTION

A method to distinguish between apoptotic and non-apoptotic cytotoxic activity of extracts from ethnobotanical plants in cancer cell lines, which cell lines include three primary human tumor cell lines, MCF-7 which is a breast cancer cell line, NCI-H460 which is a non-small cell lung cancer cell line, and SF-268 which is a CNS cancer cell line, the method comprising a sulphorhodamine proliferation assay, has been discovered.

Extracts of ethnobotanical plants, partially purified extracts of ethnobotanical plants, purified components of extracts of ethnobotanical plants, and mixtures and combinations thereof can have cytotoxic activity that is pro-apoptotic, leading to the activation of known pathways that cause programmed cell death, or that is non-apoptotic, leading to cell death by alternate pathways.

It is an advantage of this invention that compositions comprising extracts of ethnobotanical plants can be identified that are cytotoxic to cancerous cells by a mechanism of apoptosis and which are not cytotoxic to non-cancerous cells. More specifically, plants effective for such purposes are identified in natural product databases, including but not limited to the NAPRALERT database and the Chapman Hall natural product database. In a more specific aspect, active compounds include sclareol and sclareol-like compounds, and sclareolide and sclareolide-like compounds can be extracted from plants and parts thereof, and extracts formulated in treatment of cancer.

Thus, in one aspect the invention involves a method of inducing apoptosis in a living cell in a mammal. A therapeutically effective amount of a pharmaceutical composition is administered to the mammal. The composition is made up of a plant extract compound which is at least one of sclareolide, a sclareolide-like compound, sclareol, a sclareol-like compound or combinations thereof.

In another aspect, the invention involves a process for the identification of a composition or compound useful in inducing apoptosis in living cells in a mammal. The process includes an assay wherein an extract of an ethnobotanical plant is obtained. The activity of the extract is evaluated in an assay of YO-PRO-1 which exposes cells to an extract carrier combination, and measuring killing activity in cancer cells over a time. This is followed by an annexin V/PI assay performed on the YO-PRO-1 cells, and measuring killing activity in cancer cells.

In yet another aspect, the evaluation of activity of the extract is done by at least one of: detection and quantification of caspase activity; YO-PRO-1/Propicidin staining; Annexin V/Propidicum iodide flow cytometry; and Acridine orange/Ethidium bromide (AO/EtBr) staining.

### BRIEF DESCRIPTION OF THE SEVERAL VIEWS OF THE DRAWINGS

FIGS. 1-4 are plots illustrating the measurement of Apoptosis induction by Sclareol (PM 16) and Sclareolide (PM 16a) using Acridine Orange staining in the cancerous cell line, K562.

FIGS. 5-8 are plots illustrating measurement of Apoptosis induction by Sclareol (PM 16) and Sclareolide (PM 16a) using Acridine Orange staining in the cancerous cell line, U937.

FIGS. 9-12 are plots illustrating measurement of Apoptosis induction by Sclareol (PM 16) and Sclareolide (PM 16a) using Acridine Orange staining in the lympholyte cell line, R5silll.

FIGS. 13-16 are plots illustrating measurement of Apoptosis induction by Sclareol (PM 16) and Sclareolide (PM 16a) using Acridine Orange staining in the lymphocytic cell line, Molt-13.

FIG. 17 is a plot illustrating Caspase 3 activity induced by Sclareol (PM 16) and Sclareolide (PM 16a) on K562 cell line shown as a time course experiment done over twenty-four hours at 1 nm concentration.

### DETAILED DESCRIPTION OF THE INVENTION

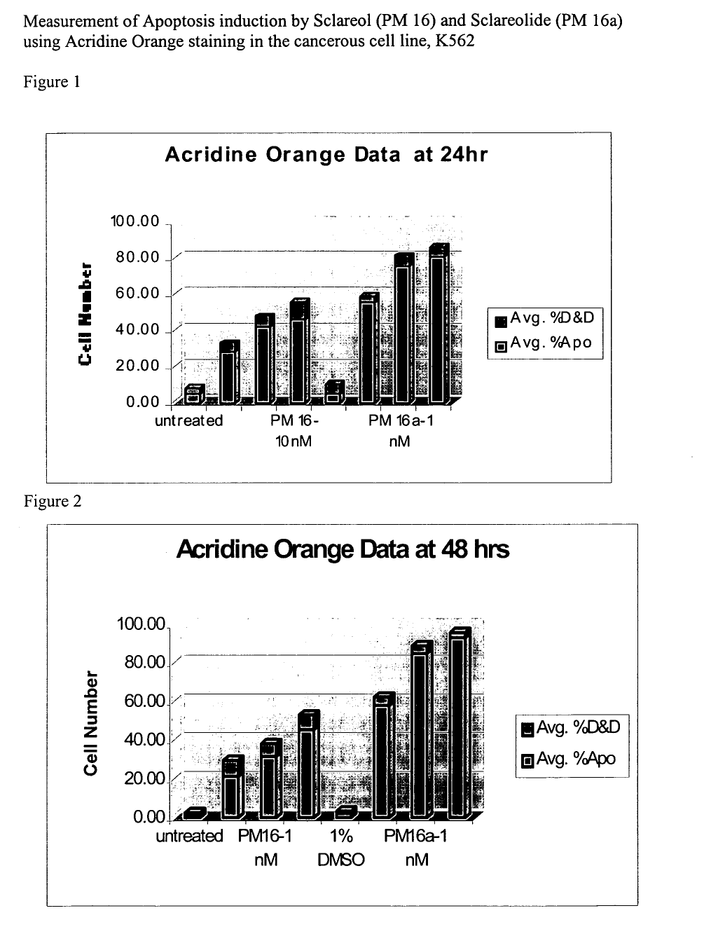

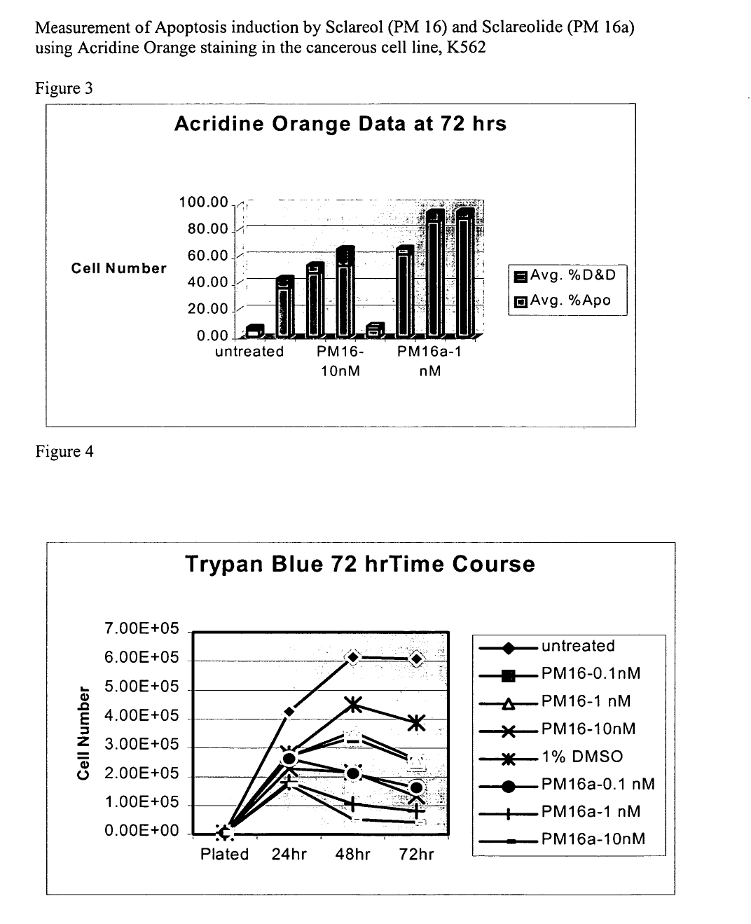

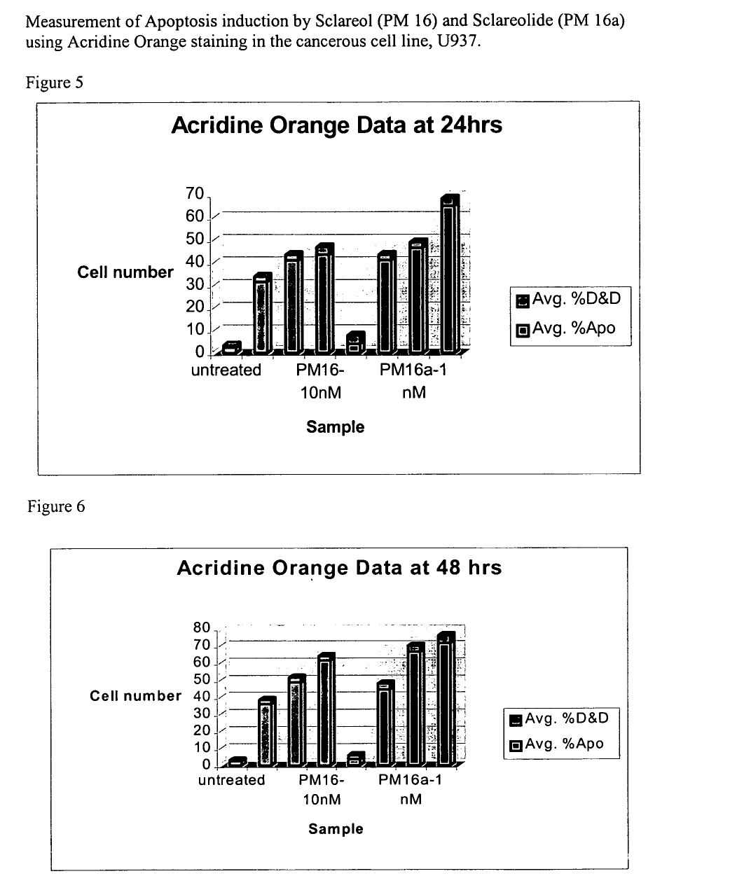

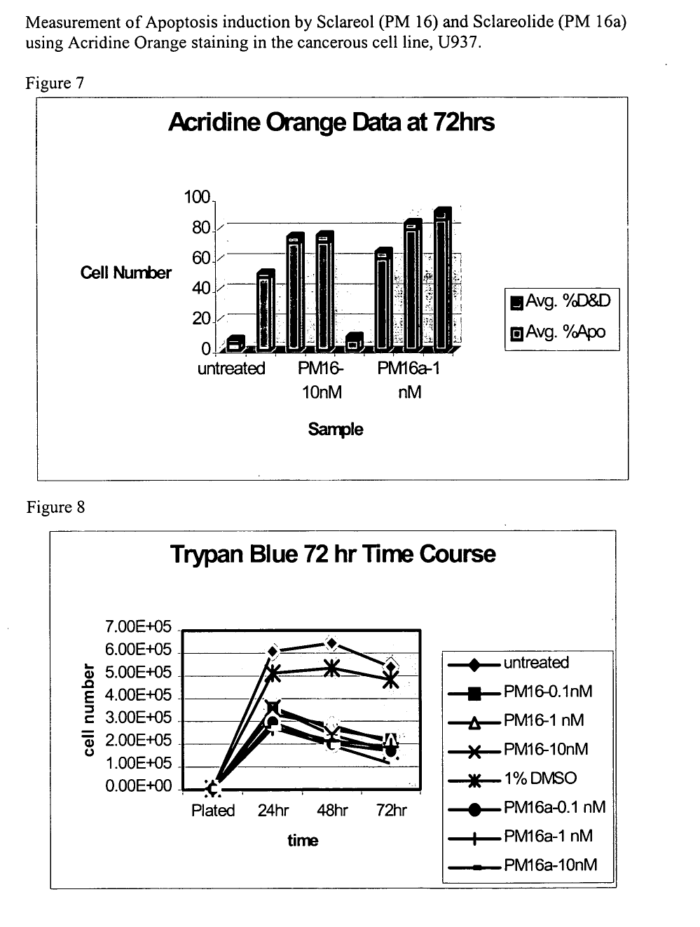

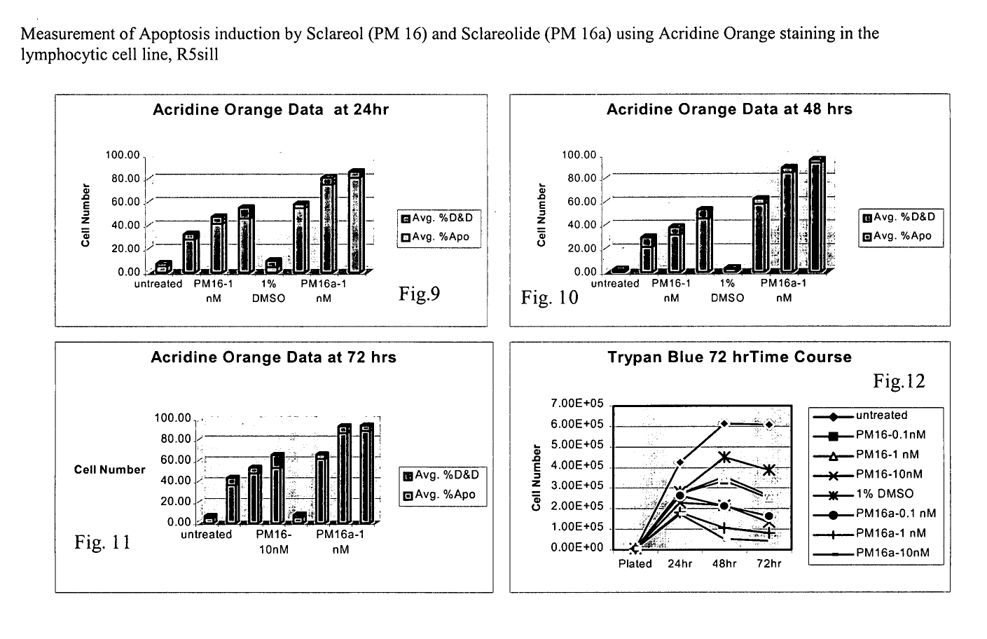

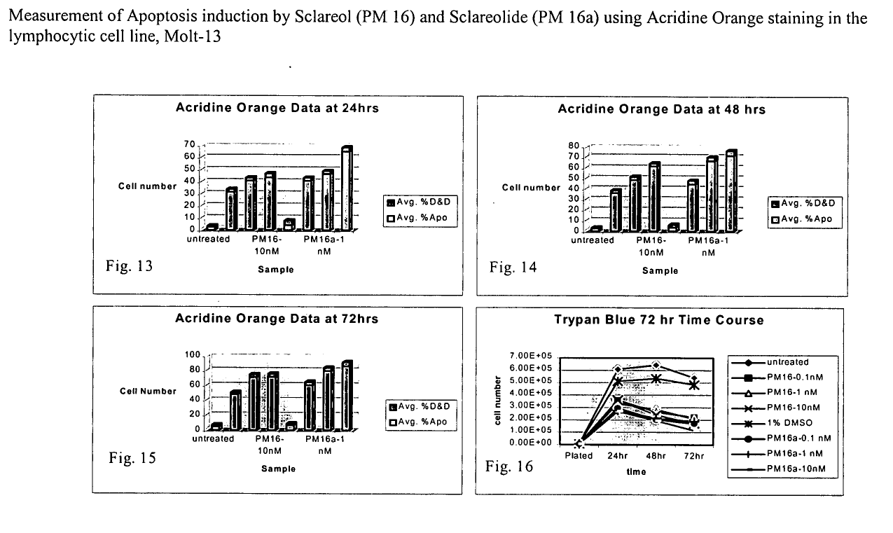

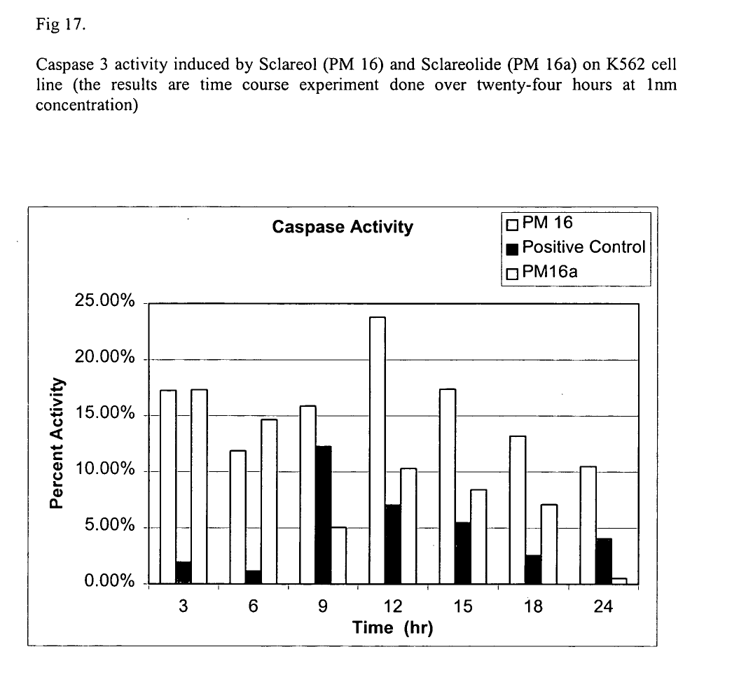

FIGS. 1-16 are a plot of changes in cell count percentage of apoptotic cells, changes in cell count percentage of necrotic cells, and in cell count percentage of live cells as percentages of total cell count of a starting cell population of K562, B cell leukemia, cells as a function of changes in concentration of the amount of plant extract or pure compounds or analogs of this invention which are applied to the total cell population. The plant extracts and pure compounds used were three botanically-related extracts or chemical analogs. The cell counts were measured 48 hours after administration of the extract to the respective samples which were maintained at about 37° C. during the time of treatment.

In one aspect of this invention, we have found that sclareol and sclareol-like compounds and sclareolide and sclareolide-like compounds can be extracted from plants and from specific parts of plants such as root, stem, leaves, bark, buds, flowers and combinations thereof, and the extracts can be formulated and used in combination with a pharmaceutically and nutraceutically acceptable carrier, such as a carrier comprising a pharmaceutically acceptable excipient and/or diluent, to provide a pharmaceutical and/or nutraceutical composition suitable for use in the treatment of cancer. In particular, we have found that sclareol and sclareol-like compounds and sclareolide and sclareolide-like compounds can be used to induce the process of apoptosis in cancer cells.

Active compounds of the present invention include sclareol and sclareol-like compounds. Sclareol-like compounds are diterpene compounds, and include, for example, sclareol, 13-episclareol, ferruginol, salvipisone, aethopisome, neoclerodane, sagequinone, romulogarzone, ortho-benzoquinone, para-benzoquinone, and clariol. Other sclareol-like compounds include abietane and icetexane diterpenoids, languidulane diterpene, paryin and pimarine diterpenes, methylene quinone diterpenoids, manoyl norditerpenoids, multicaulin, salvipimarone and pimarane diterpenoid. Additional examples of sclareol-like compounds that are useful in this invention can be identified, for example, in Gonzalez et al., Can. J. Chem. 67(2), 208-212(1989); Eanthorpe et al., Phytochem. 29, 2145-2148(1990); Kouzi et al., Helv. Chim. Acta. 73(8), 2157-2164 1990); Abraham, Phytochem. 36(6) 1421-1424(1994); Ulubelen et al. Phytochem. 36(4), 971-974 (1994); Hanson, Nat. Prod. Rep., 13, 59-71 (1996) and Topcu et al., J. Nat. Prod. 59, 734-737 (1996).

Active compounds of the present invention also include sclareolide and sclareolide-like compounds. Sclareolide-like compounds are fused-ring diterpene compounds that may be derived from sclareol by chemical or biological techniques known to those skilled in the art; and include, for example, sclareolide, ambrox, and wiedenol. Additional examples of sclareolide-like compounds that are useful in this invention can be found, for example, in Hanson, Nat. Prod. Rep. 13, 59-71 (1996); Chackalamanni et al., Tetrahedron Letters 36, 5315-5318 (1995); Barrero et al., Tetrahedron Letters 35, 2945-2948 (1994); Martres et al. Tetrahedron Letters 34, 801-8084 (1993) and Barrero et al., Tetrahedron 49(5), 10405-10412 (1993).

A preferred composition of this invention comprises an extract obtained from an ethnobiological plant.

A preferred composition of this invention includes sclareolide.

Another preferred composition of this invention includes sclarerol.

Another preferred composition of this invention includes a sclareolide-like compound.

Another preferred composition of this invention includes a sclareol-like compound.

Preferred compositions which are representative of compositions of this invention include sclareol, sclareolide, sclareol-like, or sclareolide-like compounds, examples of which compounds have the following chemical structures, and which compounds can be present in extracts from plants.

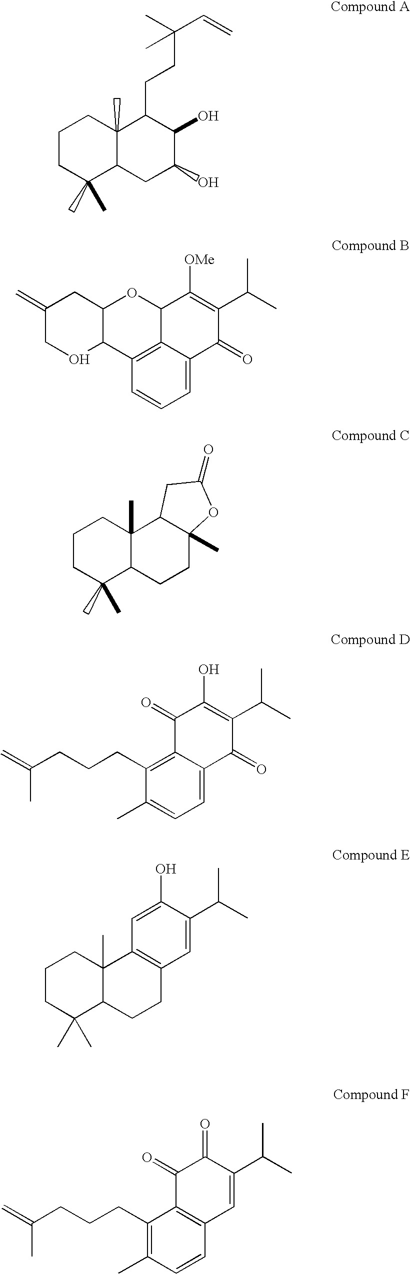

Preferred active compounds of this invention typically also include cosmetically or pharmaceutically acceptable analogs, derivatives, or salts of sclareol or sclareolide. In the practice of the present invention, the active compounds may alternatively be substituted with alkyl (both unsaturated and saturated, and branched and unbranched, such as methyl, ethyl, or isopropyl), aryl, halogen, hydroxy, alkoxy, and amino groups, as will be apparent to those skilled in the art. Additionally, any of the active compounds of the present invention may be present as an optical isomer, or chiral compound, or as a mixture of optical isomers and chiral compounds. These isomers may be isolated in pure form or enriched, for example, as a 50:50 racemic mixture of two isomers enriched to up to 100% of one isomeric pure form. Individual isomers or mixtures of isomers can be useful in this invention. The net activities of a mixture of one or more isomers will be observed in the assays of this invention.

Sclareol is an important bioactive diterpene obtained from clary sage (Salvia sclarea L.). This diterpene is not widely distributed and the most convenient sources are flower heads of clary sage plant.

Sclareol is obtained by solvent extraction of clary sage. U.S. Pat. No. 3,060,172 describes a process for the isolation of sclareol from clary sage. U.S. patent application Ser. No. 08/92,081, filed Jan. 31, 1997, and Ser. No. 08/824,147, filed Mar. 25, 1997, which applications are incorporated herein in their entirety by reference, describe additional methods of isolation of sclareol.

Sclareolide is prepared by either chemical oxidation followed by lactonization of sclareol or by biotransformation of sclareol using a yeast strain. Exemplary methods of producing sclareolide include those methods disclosed in U.S. Pat. No. 5,525,728 (to Schneider et al.), U.S. Pat. No. 5,247,100 (to Gerke et al.), and German Patent Application DE 3942358 (to Gerke et al). Briefly, these processes use a ruthenium catalyst and an oxidation step to convert sclareol into sclareolide that is present in a crude reaction product. Other exemplary methods of converting sclareol to sclareolide include the biotransformation and fermentation methods described in U.S. Pat. Nos. 4,970,163 and 5,212,078, both to Farbood et al. Sclareolide produced by these described methods is normally provided in wet or dry cake form, and is generally from about 90% to 95% pure. Sclareolide has also been reported to have therapeutic properties. See, PCT Application No. WO 06/00704 to Braquet et al. The disclosures of these patents setting forth methods of producing sclareolide from sclareol are incorporated herein by reference in their entirety.

Sclareol is a labdane diterpene (labdane-14-ene-8,13-diol) used in the fragrance industry in perfume manufacture, and also to enhance the flavor of tobacco (U.S. Pat. No. 4,441,514). Sclareol diol is chemically named decahydro-2-hydroxy-2,5,5,8a-tetramethyl-1-naphthaleneethanol. The compound is found in nature in many plant sources including Acacia sp. (Fonster et al., Phytochemistry 24:2991-1993, 1985), Salvia palestina (Phytochemistry 24:1386-1387, 1985) Stevia monardaefolia (Phytochemistry 21:2369-1371, 1982), Nicotiana glutinosa (Bailey et al. J. Gen. Microbiol. 85:57-84, 1974), and Salvia sclarea (U.S. Pat. No. 3,060,172). The latter species, also known as clary sage, represents a primary commercial source of sclareol. The sclareol produced by S. sclarea occurs in the flower stalks in the epidermal appendages or hairs known as trichomes. Although the concentration of sclareol in these hairs is relatively high, it is the primary location on the plant where sclareol is produced; there is little or no sclareol present in the leaf, root or stems of clary sage.

U.S. Pat. No. 2,905,575 describes the use of alpha-hydroxy-2,5,5,8a-tetramethyl-1-naphthaleneethanol (sclareol diol) in tobacco to impart a cedar-like aroma to the mainstream smoke.

U.S. Pat. No. 5,906,993 describes a method for treating a disorder characterized by excessive cell proliferation in a patient by administering to the patient a therapeutically effective amount of sclareolide. It also describes a method of treating excessive proliferation of benign and malignant cells in mammals comprising administering an amount of (+) sclareolide sufficient to reduce proliferation of benign and malignant cells.

(+) Sclareolide is 3aR-[3a-alpha, 5a-beta, 9a-alpha, 9b-alpha]-decahydro-3a,6,6,9a-tetramethylnaphtho[2,1-b]furane-2(1H)-one, and is a natural bicyclic terpenoid which is found for example in tobacco (Kaneko, Agr. Biol. Chem. 35(9): 1461 (1971)).

(+) Sclareolide is known for increasing or developing the organoleptic properties of food products as described in U.S. Pat. Nos. 4,917,913; 4,960,603; 4,966,783; 4,988,527; and 4,999,207. (+) Sclareolide has been used as a perfume for cigarettes (Japanese Patent 60,123,483) and as an additive to eliminate the bitter taste of coffee (U.S. Pat. No. 4,988,532). (+) Sclareolide is available from a specific number of commercial sources, for example Aldrich Chemical Co., St. Louis, Mo. (+) Sclareolide may also be prepared by synthesis, for example from (−) sclareol (Aldrich Chemical Co.) or homophamesylic acid. See for example Coste Maniere et al., Tetrahedron Letters, 29(9):1017 (1988), Mantres et al., Tetrahedron Letters 34(4):629 (1993); German Patents No. DE 4,301,555 and DE 3,942,358 and PCT Application No. WO 93/21,174.

Subbiah, in U.S. Pat. No. 6,331,551 and in U.S. Pat. No. 6,150,381, the disclosure of each of which is herein incorporated by reference, describes a cosmetic formulation for treating a skin disorder caused by a microbial infection, comprising a sclareol-like or a sclareolide-like compound in an amount sufficient to treat said skin disorder, in a cosmetically acceptable carrier, and a method of treating a skin disorder such as acne caused by a microbial infection, comprising administering a compound selected from the group consisting of sclareol and sclareolide to said subject in an amount effective to treat the disorder, wherein the microbe causing the microbial infection is a bacterium from the group consisting of Propionibacterium acnes, Enterobacter aerogens, and Bacillus subtilis.

Subbiah, in U.S. Pat. No. 5,945,546, the disclosure of which is incorporated in its entirety herein by reference, describes a method for purifying sclareolide which comprises a separation step wherein microbial cell debris is removed, and further comprises extracting an organic solution of sclareolide with an acid solution, followed by an extraction of the partially purified sclareolide with a basic solution, thus yielding sclareolide of very high purity.

U.S. Pat. No. 5,012,040 describes a somaclonal variant Nicotiana glutinosa plant and derivatives thereof which produces at least about 800 milligrams of sclareol per kilogram of fresh plant weight.

U.S. Pat. No. 4,988,527 describes the use of sclareolide for enhancing the organoleptic properties of food stuff whereby, for example, the sweetness of a jelly resulting from the use of a non-nutritive sweetener such as aspartame is enhanced by mixing sclareolide with the non-nutritive sweetener. Sclareolide is ingestibly non-toxic in the amounts used.

The compositions of this invention can be individual compounds or mixtures of individual compounds or extracts of plants or fractionated extracts of plants or purified extracts of plants, any of which can be herein referred to as a test compound.

An objective of the assays used in this invention is to discover and identify or find and isolate compounds or mixtures of compounds that can be used to induce apoptosis in cancerous cells.

Cancer cells have unique properties that allow them to proliferate and to resist apoptotic signals. Mutations that lead to cell proliferation can also cause cancer cells to be more sensitive to death stimuli. Differences between cancer cells and non-cancer cells can be exploited to trigger death preferentially in those cells that are cancerous. A method to determine such differential death ability comprises evaluating extracts in a variety of non-cancerous primary cell lines that immortalized, non-cancerous cell lines.

Apoptosis signals, or apoptosis pathways, or mechanisms of induction and progress of apoptosis can differ among cells from different sources of tissue, or tissue types, and are a function of the tissue or tissue biological function of cells in a given tissue type. Not all cells undergo the same mechanism of apoptosis. For example, in tissues such as the mammary epithelium, cells can survive because they are constantly exposed to normal and normally fluctuating levels of growth factors. In these cells, the apoptotic pathway is a default pathway that can be invoked upon removal of one or more growth factor.

Apoptosis in cells can also be induced, for example, as a result of damage to the cell, damage to organelles in the cell, damage to DNA in the cell, and combinations thereof. DNA is an acronym for deoxyribonucleic acid, usually 2′-deoxy-5′-ribonucleic acid. DNA is a code used within cells to form proteins.

The process of apoptosis can have at least three stages. One stage of apoptosis is an induction stage in which one or more diverse signals trigger or initiate the process of apoptosis in response to an induction signal. This induction of apoptosis may be reversible or it may be irreversible. A second stage of apoptosis is an effector stage in which a cell becomes irreversibly programmed for death. The effector stage is generally not reversible. A third stage of apoptosis is a degradation stage in which the cell self-destructs. The effector and degradation stages are common even between organisms as divergent as worms and mammals. The compositions of this invention can be useful to trigger at least one stage of the process of apoptosis.

Cellular mechanisms of apoptosis occurs through a number of pathways that are sometimes redundant but can also differ depending on the type of cell and the apoptotic signal, but commonalities in apoptotic pathways exist. Mitochondria appear to be necessary for apoptosis in many cells. In addition, some molecules appear to be involved in many, if not all, apoptotic pathways. These molecules include proteins of the Bcl-2 family, and cysteine proteases termed caspases.

During apoptosis, mitochondria release factors that carry out the downstream apoptotic program, including cytochrome c, a heme-containing mitochondrial protein. Cytochrome c joins two other proteins in a cytosolic complex to activate caspases. Mitochondria also release AIF (apoptosis-inducing factor), a flavoprotein that upon release translocates to the nucleus and causes chromatin condensation and DNA fragmentation through the release of a mitochondrial endonuclease. In addition, the pro-apoptotic molecule SMAC/DIABLO is released from mitochondria, causing inactivation of anti-caspase IAP proteins, allowing apoptosis to proceed. In one aspect, administration of a composition of this invention can produce activation of at least one caspase in the process of apoptosis in a cancerous cell in a patient. In another aspect, administration of a composition of this invention can result in inactivation of an anti-caspase IAP protein in a cancerous cell in a patient.

The Bcl-2 protein is the prototype of a family of both anti-apoptotic and pro-apoptotic proteins differing in their structure and their subcellular location. The Bcl-2 protein can exist predominantly as a mitochondrial outer membrane protein. Bcl-2 cam act as a mitochondrial membrane channel and maintain mitochondrial membrane integrity to prevent apoptosis. Bcl-2 can function to block release of cytochrome c. Bcl-2 can also function in the process of apoptosis downstream of cytochrome c release. Dimers can form between different members of the Bcl-2 family, and the relative abundance and phosphorylation state of each can determine whether the cell will live or die. In addition, caspases can act to modify the activity of Bcl-2 proteins during apoptosis.

Caspases are present in the cytosol and mitochondria, and exist as zymogens that can be transactivated, activated autocatalytically, or by non-caspase proteases. Caspase activation can lead to a sequential cascade that eventually leads to the degradation of cellular components including degradation of DNA. Caspases can be organized into three types as a function of their preferred substrate cleavage site. These caspase specificities suggest an order of activation in the cascade, and they are referred to as upstream activators (group III), effectors (group II), and mediators of inflammation (group I). Cellular function of at least the first two groups, I and II, can be mediators of apoptosis in many apoptotic pathways. In one aspect, administration to a patient of a composition of this invention can provide activation of at least one caspase in the process of apoptosis of a cancer cell in the patient.

Two different cell types, herein referred to as type I cells and type II cells, differ in their dependence on mitochondria for apoptosis. Upon CD95 (also called Fas) receptor ligation, both types of cells can activate mitochondria to release cytochrome c. Cytochrome c joins in a cytosolic complex with APAF-1 and dATP to activate caspase-9 which then transactivates the effector, caspase-3. However, in type I cells, strong activation of an upstream activator (caspase-8) leads directly to activation of effector caspases. Type I cells are less reliant on cytochrome c release from the mitochondria in the process of apoptosis. Type II cells require cytochrome c release from mitochondria to initiate a caspase cascade. In this scenario, cells can be typed by observing the kinetics of caspase-8 activation, and the amount of FADD, a protein associated with a complex termed DISC (death-inducing signaling complex). In addition, the ability of Bcl-2 to suppress apoptosis can be characteristic of death in type II cells.

Another type of pathway can exist in which cells in the absence of caspases undergo cell death upon CD95 ligation through mitochonodrial ROS release.

Several modes of cell death can exist. These modes of cell death include apoptosis with multiple pathways and mechanism; necrosis, in which the cell bursts; and cell death pathway intermediate between apoptosis and necrosis, but with an apoptotic-like PCD, which may or may not require caspases. In one aspect, apoptotic death can be defined as that leading to a distinct cellular morphology with chromatin condensation, blebbing of the plasma membrane and formation of apoptotic bodies, which in vivo are phagocytosed. Chromatin condensation can result from caspase-dependent activation of nucleases in the nucleus. The classical nuclease, CAD, exists only as an inactive ICAD until caspase-3 cleaves it. However, caspase-independent death, which resembles apoptosis but is morphologically different, can occur. For example, the MCF-7 cell line is caspase-3 negative, but still undergoes PCD, indicative that caspase-3 is not required for PCD in all cells that undergo apoptosis. In addition, AIF release from the mitochondria can be triggered by PARP-1 (poly-ADP ribose polymerase), an enzyme that responds to DNA damage and mediates cell death in the nucleus. Activation of PARP-1 can occur as a consequence of DNA damage, and can lead to the ADP-ribosylation of nuclear proteins. Although caspases are activated eventually, their activation is not required in all cells for apoptosis. Additionally, oxygen radicals generated by mitochondria (ROS) and low calcium levels can trigger a type of programmed necrosis, in which cells swell and nuclear condensation does not occur.

Cellular pathways that control proliferation or autonomous growth, that is, growth without external signals, and cellular pathways that control apoptosis are linked. Mutations in myc, a well-known oncogene, can cause uncontrolled proliferation, but the same mutation can trigger apoptosis. Cancer cells with a myc mutation have mutations in apoptotic pathways. Otherwise, such cancer cells would not survive and continue to be cancerous. Mutations in p53, a protein that causes cell cycle arrest and triggers apoptosis in response to DNA damage, are also required for many cancers. Greater than 50% of all tumors are known to have mutations in p53, and in certain cancers the percentage rises. Highly proliferative cancer cells are primed for apoptosis, but cannot carry out their apoptotic program because of mutations in the apoptotic pathway. In one aspect, a composition of this invention can activate the process of apoptosis in a highly proliferative cancer cell which otherwise contains at least one mutation in its genetic code that prevents activation of apoptosis in the cancer cell.

We have discovered compositions comprising novel ethnobotanical plant extracts, which extracts cause cell death differentially between normal, immortalized, non-tumorigenic cells and a variety of cancer cells. We have also discovered a series of cell-based assays useful to determine the presence of cytotoxic activity in plant extract and a method of combining these assays to find a preferred extract useful in the treatment of cancer. The method of this invention exploits the differences between cancer cells and non-cancerous cells, and in particular, the difference between mechanisms of death in cancer and non-cancerous cells.

It is an advantage of this invention that the anticancer compositions kill cancer cells preferentially in the presence of normal cells.

It is another advantage of this invention that plant extracts can be screened using assays that distinguish between apoptotic death and non-apoptotic death in several cancer cell lines.

Screenings of ethnobotanical plants were performed using a panel of cell lines comprising at least three different cell lines as representatives of major forms of human tumors. Growth inhibition and cytotoxic activity were detected by a semi automated in vitro assay. As a preliminary step, four thousand plant extracts were screened using three primary human tumor cell lines (MCF-7, a breast cancer; NCI-H460, a non-small cell lung cancer; and SF-268, a CNS cancer) in a sulphorhodamine assay which determines proliferation. Using primary human tumor cells in screening can increase the capacity for selecting a higher portion of solid-tumor drugs that can be clinically active as anticancer agents. From this screening, 290 plant extracts with the most potent activity were selected for further characterization. Positive correlations of the levels of extract activity as cytotoxic agents across at least the three cell lines were used as the criteria for selection.

In a preliminary apoptotic assay, K562 (B cell leukemia) cells were grown in wells in assay plates as recommended by ATCC (American Type Culture Collection) to 70% confluency. The cells were treated either with dimethyl sulfoxide (DMSO) vehicle alone, or with plant extracts at concentrations ranging from about 0.01 nanomolar to about 1 micromolar or about 0.1% to about 0.2% by weight of extract in DMSO. The cells in the wells were then incubated for 24 hours and then analyzed for cell death and apoptosis using an acridine orange/ethidium bromide staining assay as described herein. This assay allows quantitation or quantitative estimation of the number of dead cells and cells that have died or are dying by apoptotic and non-apoptotic mechanisms based on cell membrane permeability and condensation of nuclear chromatin. Cell numbers are then counted and the number is expressed as a percentage of the total cell population in the well used in the assay.

FIGS. 1-16 are a plot of changes in cell count percentage of apoptotic cells, changes in cell count percentage of necrotic cells, and in cell count percentage of live cells as percentages of total cell count of a starting cell population of K562, B cell leukemia, cells as a function of changes in concentration of the amount of plant extract analogs of this invention which are applied to the total cell population. The plant extracts used were three botanically-related extracts or analogs of the type indicated in the captions of the drawings. The cell counts were measured 48-72 hours after administration of the extract to the respective samples which were maintained at about 37° C. during the time of treatment. For each extract analog, increasing the concentration from 0.11 to 11 nM resulted in an increased percentage of apoptotic cells in the range from about 20% to about 95%, a decreased percentage of live cells in the range from about 5% to about 15%, and a slight increase in the percentage of necrotic cells in the range from about 5% to about 15%. Cells counts are reported as a percentage of the total cell population present at the time of measurement. A colored image of a field of cells containing a sample of total cell population after treatment with the extract shows live cells as green and having non-condensed chromatin, shows dead apoptotic cells as orange and having condensed chromatin, and shows necrotic cells as orange and having non-condensed chromatin. A control experiment using the same live K562, B cell leukemia, cells but treated with DMSO vehicle alone provides a color image which shows substantially all of the cells in the population to be alive after treatment with the DMSO vehicle and does not show any appreciable cell death by apoptosis or by necrosis in the population as a result of the DMSO treatment. This assay indicates that an extract of this invention can kill at least one cell and preferably from about 50% to about 75% of the cells, and more preferably up to all cells in a population of cancer cells with a primary mechanism of death of the cancer cells as apoptosis.

K562, Caco-2, and MCF-7 cells used in this invention are maintained as recommended by the ATCC.

K562 cells (ATCC CCL-243) are human hematopoietic malignant cells derived or removed from a patient with chronic myelogenous leukemia (CML). These cells closely resemble B cells and can serve as an in vitro experimental model of CML.

Caco-2 cells (ATCC HTB-37) are human colon cancer cells that can serve as an in vitro experimental model for colorectal cancer.

MCF-7 cells (ATCC HTB-22) are human, estrogen receptor-positive breast cancer cells which can serve as an in vitro model for breast cancer.

Each of the K562 cell line, the Caco-2 cell line, and the MCF-7 cell line is tumorigenic in mice and each can be used for in vivo studies.

MCF-10A cells are human breast cells, which are non-tumorigenic in immunosuppressed mice and which can serve as an in vitro model for “normal” breast cells. These cells are originally from the Karmanos Cancer Institute (Detroit, Mich.). These cells are maintained in Dulbecco minimum essential medium/Ham's F12 medium (DMEM/F12) supplemented with 10% fetal bovine serum (FBS), hydrocortisone and epidermal growth factor (EGF). DMEM/F12 is a serum-free medium formulation for general use, and is a 1:1 blend of DMEM and Ham's F12 media supplied complete, ready-to-use with L-glutamine, Hepes, BPE and EGF for culturing a wide range of cell types. It contains no phenol red or antibiotics.

We have stably expressed the bcl-2 pro-apoptotic gene in MCF-10A cells, a non-cancerous immortalized cell line to determine the pathway of apoptosis triggered by growth factor withdrawal in the parent cells, to determine whether or not mitochondria are involved, and to determine whether or not apoptosis is altered upon bcl-2 over-expression. We have found that bcl-2 expression changes the mitochondrial membrane potential of the cells, and also causes a slow-growth phenotype. Apoptosis triggered by growth-factor withdrawal is not altered upon bcl-2 expression. While still uncertain, it is possible that because bax is concomitantly overexpressed with bcl-2, Bax prevents the anti-apoptotic activity of Bcl-2 in this model system.

Assays

In the processes and assays of this invention, all reagents are from Sigma-Aldrich Chemical Company unless otherwise indicated. All assays are performed at least two separate times, and at each time each assay is run in triplicate.

Caspase Detection:

The Caspase detection kit (Oncogene) is used to detect and quantify caspase activity as an indication of apoptosis induction. Cells are grown in tissue culture dishes, the plant extracts of this invention are added and the cells are allowed to incubate. Time points at which evaluations of the cells stati are made are taken at 6, 12, 24, and 36 h. Untreated cells are used as negative controls. Cells treated with the apoptosis inducer staurosporine can serve as positive controls. The cells are transferred to microfuge tubes, FITC-VAD-FMK is added and allowed to incubate for 30 minutes, after which time the cells are pelleted by centrifugation and the supernatant liquid above the cells is discarded. Cells are then washed with PBS and subsequently transferred to wells in 96-well microtiter plates at a concentration of 5,000-10,000 cells/well. Fluorescence emissions from the wells in the plates are detected and quantified using a fluorescent plate reader (FL-600, Biotek Instruments, Inc.) with excitation and emission filters of 485 nanometers (nm) and 535 nm, respectively.

YO-PRO-1/Propidium Iodide Staining:

YO-PRO-1 nucleic acid stain, available from Molecular Probes as Y-3603, forms the basis of an assay for apoptotic cells that is compatible with fluorescence microscopy.

Propidium iodide (PI) is a cell-impermeant dye and is not taken up by cells during the initial stages of apoptosis. Later stages of apoptosis are accompanied by an increase in membrane permeability, which allows propidium iodide to enter cells.

Cells are grown in 96-well black microtiter plate and the extracts added. Time points of 12 to 48 h. are taken. An optimized amount or ratio of dye to substrate is added and the plates are incubated in the dark for 30 min. Negative control wells without dye and without cells are used to determine background. Positive controls are treated with staurosporine to induce apoptosis. The plates are read in a fluorescent plate reader (FL-600, Biotek Instruments, Inc.) with excitation and emission filter of 485 nm and 535 nm, respectively for YO-PRO-1 and with excitation and emission filter of 520 nm and 595 nm, respectively for propidium iodide. The YO-PRO-1 emission intensity value is divided by the PI emission intensity value to obtain a ratio of the number of cells which die as a result of apoptosis to the number of cells which die as a result of necrosis.

Annexin V/Propidium Iodide by Flow Cytometry:

The ApoTarget Annexin-V FITC Apoptosis Kit (from Biosource International) is used as follows. Cells are grown in tissue culture plates and treated with the extracts of this invention at time points as described above, then washed and harvested. The cells are resuspended in PBS buffer at pH 7.0; two dyes, Annexin V and Propidium iodide, are added to the cells; and the cells are incubated for 15 minutes in the absence of room light. The cells are then analyzed by flow cytometry with excitation at 488 nm. Positive and negative controls are prepared as described for the YO-PRO-1/PI assay.

Four types of cells are distinguished in this Annexin V/Propidium iodide assay:
cell type A1 is unstained and represents live, non-apoptotic cells;
cell type A2 is Annexin-positive and is PI negative and represents apoptotic cells;
cell type A3 is Annexin-negative and PI-positive and represents dead, non-apoptotic cells; and
cell type A4 is Annexin-positive and PI-positive and represents dead cells which cannot be distinguished as apoptotic or non-apoptotic cells.

Acridine Orange/Ethidium Bromide (AO/EtBr) Staining:

Subconfluent cells are grown in 24 well plates and treated with the extracts with timepoints and controls as described for the YO-PRO-1 assay. The cells are harvested and washed, and resuspended at 3 to 5×10⁶ cells/ml with 4 μg/ml each AO (Acridine Orange) and EtBr (Ethidium bromide) in PBS (phosphate buffered saline solution, pH 7.4). The cells are placed on ice and covered to protect from ambient light. The cells are viewed by fluorescence microscopy with a 20× or 40× objective and a FITC filter, and 100 cells of each sample are counted. Live cells fluoresce green and dead cells fluoresce orange. Apoptotic cells are distinguished by the presence of condensed nuclear chromatin.

The fraction of cells which are dead or dying is equal to (the number of live apoptotic cells plus the number of dead apoptotic cells plus the number of dead non-apoptiotic cells) divided by (the total number of cells counted).

The fraction of apoptotic cells in the dead or dying cell population is equal to (the number of live apoptotic cells plus the number of dead apoptotic cells) divided by the total number of cells counted.

In accordance with the invention the mechanism of death in cancer cells caused by compositions made up of plant extracts of this invention is determined. The extracts are obtained from plant sources, and the extracts have cytotoxic activity.

In accordance with another aspect of the invention the efficacy of compositions including plant extracts of this invention in the induction of apoptosis in cancer cells is quantified.

Representative and non-limiting selections of ethnopharmacological plant species that are useful in this invention as sources of extract materials, which extracts can contain compounds that exhibit apoptotic induction activity in diseased cells can be selected from the group consisting of Acacia farnesiana, Acacia sinuata, Achyranthes aspera, Ageratum conyzoides, Alangium salvifolium, Allium cepa, Amaranthus spinosus, Amorphophallus paeoniifolius, Anthocephalus chinensis, Ardisia solanaceae, Artocarpus integrifolia, Asclepias curasavica, Asparagus racemosus, Atalantia monophylla, Baliospermum montanum, Bauhinia pupurea, Bauhinia tomentosa, Bauhinia variegata, Bidens bipinnata, Bixa orellana, Boerhaavia diffusa, Bombax ceiba, Boswellia serrata, Buchanania lanzan, Bulbostylis barbata, Calotropis gigantea, Capparis zeylanica, Careya arborea, Cassia fistula, Cassia occidentalis, Cassia tora, Cassine glauca, Cedrus deodara, Chomaesyce hirta, Chomaesyce prostrata, Cissampelas pareira, Cissus pallida, Cissus quadrangularis, Clerodendrum serratum, Coccinia indica, Conyza canadensis, Cordia myxa, Coriandrum sativum, Crataeva religiosa, Croton sparsiflorous, Cryptolepis buchanani, Curculigo orchioides, Cyamopsis tetragonoloba, Cyperus rotundus, Datura innoxia, Datura metel, Dolichandrone crispa, Embelia ribes, Erythrina indica, Erythrina stricta, Eupatorium odoratum, Ficus benghalensis, Ficus religiosa, Gardenia latifolia, Glycosmis arborea, Gmelina arborea, Grangea sp., Gymnema sylvestre, Hemidesmus indicus, Heteropogon contortus, Ichnocarpus frutescens, Indoneesiella echiodes, Ipomoea hederifolia, Kalanchoe pinnata, Lannea coromandalica, Leucas aspera, Luffa acutangula, Madhuca indica, Mallotus phillipensis, Melochia corchorifolia, Melothria sp., Mesua nagassarium, Mimosa pudica, Moringa oleifera, Mucuna pruriens, Nerium indicum, Nyctanthes arbor-tristis, Ocimum americanum, Ocimum tenuiflorum, Opuntia monocantha, Oroxylum indicum, Oxalis corniculata, Pandanus fascicularis, Pergularia daemia, Phyllanthus acidus, Physalis minima, Piper longum, Plantago ovata, Polycarpea corymbosa, Polygala erioptera, Polygonum barbatum, Pongamia glabra, Rhus succedanea, Sapindus laurifolius, Sarcostemma acidum, Sida acuta, Smilax zeylanica, Solanum torvum, Solanum trilobatum, Strychnos nux-vomica, Tamarindus indica, Tephrosia purpurea, Tephrosia tinctoria, Terminalia bellirica, Thottea siliquosa, Tinosporia cardifolia, Tragia connabina, Tragia involucrata, Trichopus zeylanicus, Vetiveria zizaniodes, Vitex altissima, Wattakaka volubilis, Xanthium indicum, Ziziphus oenoplia, Amorphophallus paeoniifolius, Cyamopsis tetragonoloba, Coccinia indica, Physalis minima, Calotropis gigentia, Trichopus zeylanicus, Solanum nigrum, Boerhavia diffusa, Indigofera tinctoria, Sida acuta, Anisomeles malabarica, Merremia tridenta, Sida cordifolia, Calotropis procera, Alpinia galangal, Euphorbia hirta, and combinations thereof.

Compositions of aliquots of extracts of these plants can contain one or more compounds that can induce apoptosis in diseased cells. Compounds that can induce apoptosis in diseased cells are sometimes referred to herein as apoptotic agents. Aliquots of extracts from two or more plants can be combined and fractionated to provide additional combinations of compounds as mixtures, which mixtures can contain one or more compounds that can induce apoptosis in diseased cells or apoptotic agents. Combinations of apoptotic agents prepared according to this method can exhibit apoptotic behavior with respect to cancer cells. Individual apoptotic agents and mixtures of apoptotic agents can be isolated by chromatographic methods or optionally chemically modified and isolated to provide novel apoptotic agents that constitute compositions of this invention.

Extracts obtained according to this invention can be subjected to immediate assay for apoptotic agent activity (i.e., assayed to demonstrate that the apoptotic agent can induce apoptosis in cells, particularly in diseased cells such as cancer cells) by the methods of this invention. Individual components of the extract materials can be purified and isolated as pure compounds that exhibit apoptotic agent activity. Alternatively, mixtures of compounds can be isolated from the extract materials, wherein at least two components of the mixture exhibit apoptotic agent activity. In one aspect, for example, a mixture of apoptotic agents can produce an arithmetically additive efficacy in the amount of induction of apoptosis produced by the mixture, wherein the amount of apoptosis induced is a linear function of the concentration of each component and the amount of apoptosis induced by the mixture is the sum of the amount of apoptosis induced by each component of the mixture.

In another aspect, for example, a mixture of apoptotic agents can produce a synergetically additive efficacy in the amount of induction of apoptosis produced by the mixture, wherein the amount of apoptosis induced is a non-linear function of the concentration of each component and the amount of apoptosis induced by the mixture is greater than the sum of the amount each separate component in the induction of apoptosis. The amount of an apoptotic agent in a mixture can range from about 0.1% by weight to about 99.9% by weight of the mixture of apoptotic agents. Alternatively, a mixture of a compound that exhibits apoptotic agent activity together with compounds that do not exhibit apoptotic agent activity can be isolated from the extract material. Crude extract materials can be assayed or screened for apoptotic agent activity, or individual components can be screened for activity.

Alternatively, extract material obtained according to this invention can be oxidized before it is subjected to the assay of this invention to screen for apoptotic agent activity. Oxidation can be accomplished by exposing the extract material to oxidizing conditions. Representative oxidizing conditions include exposure of the extract material to oxygen gas particularly when the extract material is dissolved in a solvent or suspended in a solvent; by exposure of the extract material to oxygen in air particularly when the extract material is dissolved in a solvent or suspended in a solvent; by exposure of the extract material to hydrogen peroxide in water or a mixture of water and a compatible organic solvent such as methanol or ethanol or by phase transfer oxidation conditions known in the art; by exposure of the extract material to organic peracidics such as peracetic acid and perphthalic acid, particularly when the extract material is dissolved in a solvent such as methylene chloride or suspended in a solvent such as water; by exposure of the extract material to inorganic peracids or inorganic peracid salts such as sodium persulfate, sodium perborate, sodium perchlorate, particularly when the extract material is dissolved in a solvent or suspended in a solvent such as water or a combination of alcohol and water; and by exposure to singlet oxygen generated by sensitized irradiation, particularly when the extract material is dissolved in a solvent or suspended in a solvent. Irradiation useful for singlet oxygen generation from triplet oxygen in the presence extract material, optionally dissolved in a solvent such as methylene chloride, can be that emitted from ultraviolet and/or from visible light sources or from incandescent light sources.

In one aspect, one or more components of the extract material can act as a sensitizing agent for singlet oxygen generation in the presence of light. Alternatively, a known singlet oxygen-sensitizing agent such as a benzophenone can be added to the extract or to a solution or suspension of the extract material in the presence of oxygen and irradiation to generate singlet oxygen. Extract materials that are oxidized by exposure to oxidizing conditions can contain additional chemical functional groups such as epoxide groups, alcohol groups, diol groups, vicinal cis-diol groups, vicinal trans diol groups, allylic alcohol groups, carboxylic acid groups, aldehyde groups, and other functional groups such as acetate or other ester groups that are not originally present in the extract materials isolated from natural sources. Additional oxidizing conditions such as treatment with halogens, halogen oxides, nitric oxides, nitrate esters, and acetyl nitrate can introduce additional functional groups into the extract materials.

The process of this invention involves an extraction of an ethnopharmacological plant. The process can further involve at least one chemical modification step performed on an aliquot of the extract or on an isolated component of the extract or a mixture thereof. For example, the chemical modification step can be selected from the at least one of oxidation, reduction, esterification, amidation, hydrolysis, and alkylation, and combinations thereof.

Extracts of an ethnopharmacologic plant and components of such extracts of this invention can be obtained from a single plant or a mixture of plants. Extracts can be obtained from any part of an ethnopharmacologic plant or combinations of parts of the plant, for example, an entire ethnopharmacologic plant, or from the group consisting of a root thereof, a bark thereof, a stem thereof, a leaf thereof, a sap thereof, a branch thereof, a fruit thereof, a flower thereof, a trunk thereof, and combinations thereof.

A plant or plant part such as a root is pulverized into a powder and is extracted with an organic solvent. Useful solvent classes include but are not limited to ether, alkane, aromatic, ester, aralkane, ketone, halogenated alkane, sulfoxide, amide, nitrile, alcohol, supercritical fluid, liquefied petroleum, and combinations thereof. Useful solvents include, for example, a solvent selected from the group consisting of diethyl ether, petroleum ether, hexane, toluene, acetone, acetonitrile, tetrahydrofuran, ethyl acetate, methylene chloride, chloroform, isopropanol, supercritical carbon dioxide, supercritical dimethly ether, liquefied propane, and combinations thereof. The solvent can be removed by evaporation using heat and pressure change conditions to concentrate the extract. Optionally, a solution of the extract in a water insoluble solvent can be washed or extracted with a basic solution such as saturated sodium carbonate, saturated sodium bicarbonate, or a solution containing sodium or potassium hydroxide at pH 8 to 14. Thereafter, the water insoluble solvent can be dried using sodium sulfate or magnesium sulfate, filtered, and the solvent evaporated. The extract can be chromatographed to obtain individual fractions that can be evaluated for apoptotic agent activity.

An apoptotic agent according to this invention above can be formulated for administration in a pharmaceutically acceptable carrier in accordance with known techniques, for example, those described in Remington, The Science And Practice of Pharmacy (9th Ed. 1995) that is incorporated herein by reference in its entirety.

In the preparation of a pharmaceutical formulation according to the invention, an extracted component or mixture of components or chemically modified component which can include one or more physiologically acceptable salts thereof is typically admixed with, inter alia, a pharmaceutically acceptable carrier. The carrier may be a solid or a liquid, or both, and is preferably formulated with the apoptotic agent as a unit-dose formulation, for example, a tablet or an injectable suspension or an injectable solution, which may contain from 0.01 or 0.5 percent to 95 percent or 99 percent by weight of the extracted component or mixture of components or chemically modified component.

The method of administration of a formulation of this invention can be selected from the group consisting of oral, rectal, topical, buccal, sub-lingual, vaginal, parenteral, subcutaneous, intramuscular, intradermal, intravenous, topical, transdermal, transmucosal, inhalation, and combinations thereof. The most suitable route in any given case will depend on the nature and severity of the condition being treated, particularly when the condition is cancer. When the cancer is systemic, an injectable formulation can be preferred. When a solid tumor is present in a tissue, an injectable formulation can be preferred. Other preferred formulations comprise topical and inhalation formulations.

The compounds of this invention can be formulated in pharmaceutically acceptable dosage forms such as for injectable use, for oral use, for inhalation use, for transdermal use, for transmembrane use, and the like. Formulations suitable for oral administration may be presented in discrete units or dosage forms, such as capsules, cachets, lozenges, tablets, pills, powders, granules, chewing gum, suspensions, solutions, and the like. Each dosage form contains a predetermined amount of the extracted or extracted and chemically modified apoptotic agent of this invention. Solutions and suspensions can be in an aqueous or non-aqueous liquid or as an oil-in-water or water-in-oil emulsion.

Formulations of an apoptotic agent of this invention may be prepared by any suitable method of pharmacy. A preferred method comprises the step of bringing into association, for example by mixing, by dissolution, by suspension, by blending, by granulation, and the like an extract or component of an extract of an ethnopharmacologic plant, optionally and sometimes preferably as a component of the extract in purified form, and a pharmaceutically acceptable carrier such as a liquid, for example a liquid consisting of water, an aqueous solution of a pharmaceutically acceptable alcohol, a pharmaceutically acceptable oil such as an edible oil such as a triglyceride or mixture of triglycerides of natural sources such as an edible plant oil, an emulsion of a pharmaceutically acceptable oil in an aqueous medium comprising water, and which aqueous medium may contain one or more pharmaceutically acceptable excipients such as an excipient selected from the group consisting of a pH buffering agent, a matrix forming sugar, a pharmaceutically acceptable polymer, a pharmaceutically acceptable tonicity modifying agent, a surface modifier or surfactant useful to form micelles or to form liposomes or to form emulsions. The extract or component can also be combined in solid form with pharmaceutically acceptable excipients such as ingredients used in tablet formation such as release agents and compressing agents, silica, cellulose, methyl cellulose, hydroxypropylcellulose, polyvinylpyrolidinone, gelatin, acacia, magnesium stearate, sodium lauryl sulfate, mannitol, lactose, colorants, dyes, and formed into a dosage form such as a tablet, capsule, caplet, pill, powder, granule, and the like. Optionally, the tablet or related dosage form can be coated with a polymer coating such as an enteric and/or moisture barrier polymer coating such as can be applied by spraying, spray drying, or fluid bed drying methods.

The extract or component can be combined in an aqueous or aqueous-organic, or an organic liquid solvent together with one or more pharmaceutically acceptable excipient and then dried, for example by spray drying, lyophilization, fluid bed drying, or evaporation to form a solid in which the component or extract is imbibed or uniformly dispersed or suspended. The formulations of the invention can be prepared by admixing, preferably by uniformly and intimately admixing, an extract or component of an extract of an ethnopharmacologic plant, optionally and sometimes preferably in purified form, with a liquid or with a finely divided solid carrier or matrix-forming excipient or mixture of excipients, then, if necessary, shaping the resulting mixture into a dosage form. For example, a tablet may be prepared by compressing or molding a powder or granules or granulates containing an isolated extract of this invention, optionally with one or more accessory ingredients. An isolated extract can also mean a chemically modified isolated extract. Compressed tablets may be prepared by compressing in a tablet press a mixture of an extract of this invention or component thereof together with one or more pharmaceutically acceptable excipitent materials, which mixture can be in a free-flowing form such as a powder or granules optionally mixed with a pharmaceutically acceptable material selected from the group consisting of a binder, a lubricant, an inert diluent, a surface active agent, a dispersing agent, and combinations thereof. Molded tablets may be made by molding, in a tablet mold machine, a solid powdered mixture of an extract or component of an extract of this invention together with one or more pharmaceutically acceptable excipient, which mixture is moistened with an inert liquid binder such as water or alcohol.

A formulation suitable for buccal or sub-lingual administration to a patient in need of treatment by an apoptotic agent of this invention includes a lozenge such as a lozenge comprising an isolated extract or purified component thereof of this invention in a flavored base such as sucrose, acacia, tragacanth, and the like; and a pastille comprising an extract of this invention or a component thereof in an inert base such as gelatin, glycerin, sucrose, acacia, and the like.

The concentration of the apoptotic agent in a dosage form containing an antagonist of this invention depends on the activity and bioavailability of the apoptotic agent, and it is at least a therapeutically effective amount of apoptotic agent, preferably from 0.01% by weight to about 50% by weight of the dosage form, more preferably from 0.1% to 40% by weight. Additional concentrations can be selected from 0.1% to 5% by weight, 0.1% to 10% by weight, 0.1% to 20% by weight, 1% to 10% by weight, and 1% to 15% by weight of the dosage form. Depending on the dosage form, pharmaceutically acceptable excipients make up the remainder of the dosage form weight. Excipients such as sugars (lactose, mannitol, sucrose, and the like; polymers such as polyvinylpyrrolidone, poly(vinyl alcohol), pharmaceutically acceptable cellulose derivatives, silica, are useful in solid oral dosage forms.

A formulation of the present invention that is suitable for parenteral administration can comprise a sterile aqueous solution, and a non-aqueous solution in an organic solvent safe for injection of the isolated extracted apoptotic agent of this invention. Useful injectable dosage forms containing an apoptotic agent of this invention preferably are isotonic with the blood of the intended recipient. Tonicity of the dosage form can be adjusted and/or maintained by addition of pharmaceutically acceptable for injection water-soluble excipients such as sugars, buffer salts, and combinations thereof. These dosage forms may optionally contain antioxidants, buffers, bacteriostats, and dissolved solutes that render the formulation isotonic with the blood of the intended recipient. Aqueous and non-aqueous sterile suspensions may include pharmaceutically acceptable suspending agents and thickening agents. Formulations of this invention can be presented in unit-dose or multi-dose containers. For example, for injectable use, a formulation can be sealed in an ampoule or vial, preferably sealed in oxygen-free form such as in a vial under an inert oxygen-free gas such as nitrogen or argon or other non-reactive gas, or a mixture thereof. In another embodiment, a dosage form of this invention may be stored in a freeze-dried or lyophilized form containing a small quantity of water, for example from 0.01% to about 5% by weight of the dried dosage form, which dosage form then requires only the addition of a sterile liquid carrier, for example, isotonic aqueous saline solution, and optionally buffered to between about pH 5 to pH 9, or by addition of water-for-injection immediately prior to use. Extemporaneous injection solutions and suspensions may be prepared from sterile powders, granules and tablets of the kind previously described.

A formulation of this invention containing an apoptotic agent and which is suitable for rectal administration is preferably presented as a unit dose suppository. A suppository dosage form containing an apoptotic agent of this invention may be prepared by admixing an isolated extract of this invention with one or more conventional pharmaceutically acceptable solid carriers, for example, such as cocoa butter, to form a mixture containing the apoptotic agent, and then shaping the resulting mixture.

A formulation of this invention suitable for topical application to skin preferably can be in the form of an ointment, a cream, a lotion, paste, gel, spray, aerosol, oil, or a combination thereof. A pharmaceutically acceptable carrier in this embodiment can be selected from the group consisting of petroleum jelly, lanoline, a polyethylene glycol, a polyethylene glycol ether or ester, an alcohol, a transdermal penetration enhancer, and combinations thereof.

A formulation of this invention suitable for transdermal administration of an apoptotic agent of this invention may be presented as a discrete patch dosage form. The patch can be adapted to remain in intimate contact with the epidermis or stratus corneum of a recipient for a prolonged period of time such as from 8 hours to about 48 hours or longer. A formulation suitable for transdermal administration can also be delivered by an iontophoretic delivery mechanism such as by using an applied voltage difference between two portions of the dosage form, each of which is in contact with the skin of a patient.

A therapeutically effective dosage of any apoptotic agent of this invention isolated from an extract of a plant, the use of which is in the scope of present invention, will vary from one apoptotic agent compound to another apoptotic agent compound, and from patient to patient, and will depend upon factors such as the age of the patient and the diagnosed condition of the patient and the route of delivery of the dosage form to the patient. A therapeutically effective dose and frequency of administration of a dosage form can be determined in accordance with routine pharmacological procedures known to those skilled in the art. Dosage amounts and frequency of administration can vary or change as a function of time and particular condition being treated. For example, a dosage of from about 0.1 to 1000 mg/kg, preferably from about 1 to about 100 mg/kg, may be suitable for treatment of a cancer such as breast cancer.

In one aspect, a preferred dosage of an apoptotic agent of this invention can be from about 20 to about 35 mg/kg to have therapeutic efficacy.

In one aspect, an apoptotic agent of this invention can be in the form of a salt, such as a protonated amine form or a deprotonated carboxylate or other acid form. Intravenous dosage forms can sometimes be up to about 20 mg/kg of apoptotic agent. A preferred dosage from about 30 mg/kg to about 50 mg/kg may be employed for oral administration. A preferred dosage from about 20 mg/kg to 30 mg/kg may be employed for intramuscular injection. The frequency of administration of a dosage form of this invention can be once, or twice, or three times, or four times per day. A useful duration of treatment of a patient can be from about one or two days, up to five or seven days, up to two or three weeks, or until symptoms of a disease state in a patient are essentially controlled. An apoptotic agent of this invention isolated by extraction can be used in the treatment of diseased states such as cancer, fungal infection, bacterial infection, acne, eczema, psoraisis, and the like.

Preparation and Fractionation of Plant Extracts

Compositions comprising extracts from plants are isolated from the following list of plants by extraction with a suitable solvent. A suitable solvent can comprise, for example but not limited to: water; an alcohol having from 1 to about 6 carbon atoms such as methanol, ethanol, isopropanol, butanol and the like; a halogenated alkane or a halogenated alkene having from 1 to about 10 carbon atoms and one or more halogens such as methylene chloride, chloroform, carbon tetrachloride, trichloroethylene, trichloroethane, and the like, a liquefied gas such as liquid propane, a supercritical fluid such as supercritical carbon dioxide, supercritical dimethyl ether, and the like; a ketone containing from 3 to 10 carbon atoms, such as acetone, methyl ethyl ketone, and the like; pyridine; DMSO; amides such as DMF and HMPA; and combinations thereof. The solvent is evaporated. Each extract is fractionated by solid phase extraction (SPE) as follows. The crude extracts are fractionated using a solid phase extraction protocol. Solid phase extraction involves gel cartridges with different matrices (silica or C18 gels). The extracts are eluted onto and adsorbed on to the gel. The extracts are then eluted with solvents of increasing polarity to separate each crude extract into at least ten semi-purified fractions, each of which are eluted and collected separately. This process is useful for enriching the relative purity of each fraction as well as removing from each component of the extract compounds which do not elute from the adsorbent under the solvent and time conditions used. Subsequently, each of these extracts are analyzed on high pressure liquid chromatography (HPLC; HP1100 with DAD detector) for chemical profiling and analysis. An aliquot of each of the fractions is transferred to a well in a 96-well microtiter plate, the solvent is removed, and the extract component in the well is used for the assays.

Screening for Induction of Cell Death

A YO-PRO-1/PI staining assay is performed as a first screening assay on fractions of plant extracts in order to quantify the number of dead cells produced by each of the fractions and to determine whether cell death occurs via an apoptotic or a non-apoptotic mechanism. A caspase activity assay is then used as a second assay to quantify apoptotic cell death induced by each of the extract fractions. Both assays are performed in a high-volume format using microtiter plates and/or flow cytometry.

First Assay

YO-PRO-1 is available from Molecular Probes as Y-3603, and is a fluorescent intercalating DNA dye. YOPRO-1 does not enter live cells, but stains DNA in cells undergoing apoptosis because their membranes become slightly permeable, although the cell is still intact. PI only enters dead cells which have a highly permeable membrane. Therefore, this assay distinguishes dead from live cells and also identifies the mechanism of death as apoptotic or necrotic, based on membrane permeability. The ratio of the dyes is optimized to minimize the masking effect of each dye on the other's emission signal properties. An advantage to this assay is that it is useful with high-throughput evaluations. Adherent cells are cultured in and the assay is performed with the same plate. This assay is a cell viability or death assay that also indicates one or more mechanism of cell death.

Extract fractions that produce a killing effect on cells, which effect is indicated in the YO-PRO-1/PI assay, are subjected to an annexin V/PI staining assay which is an alternate assay for cell death and apoptosis, to verify the results obtained from the YO-PRO-1/PI assay. The annexin V/PI staining assay employs fluorescein-labeled annexin-V (annexin-V FITC) in concert with PI. Flow cytometry is used to detect cells undergoing apoptosis. During the early stage of apoptosis, cells begin to display phosphatidylserine (PS) on their cell surface membranes. Phosphatidylserine is readily detectable by staining the cells with annexin-V FITC. The plasma membrane becomes increasingly permeable during the later stages of apoptosis. The plasma membrane is also permeable in cells that experience necrotic cell death. PI can cross permeable cell membranes and bind to DNA. The fluorescence emission provides a means to identify an reduction in or loss of membrane integrity that is associated with necrosis and/or late stages of apoptosis.

As an alternative, an acridine orange (AO)/ethidium bromide (EtBr) staining assay can be used with fluorecence microscopy to quantify and determine the mechanism(s) of cell death. This assay is similar to annexin V FITC/PI assay in that it distinguishes between apoptotic cell death and necrotic cell death.

Acridine orange and ethidium bromide are both nucleic acid stains used to detect if nuclear chromatin is condensed, which is indicative of apoptosis, or non-condensed which is indicative that apoptosis has not progressed to a detectable stage or is absent. Acridine orange can enter all cells and fluoresces with green emission. Ethidium bromide enters dead cells in which the cell membrane has become highly permeable. Live cells, which are permeable only to AO, fluoresce green, but dead cells, which are permeable to both AO and EtBr, fluoresce orange due to the dominance of the EtBr. The AO/EtBr assay allows quantitation of four cell types based on cell membrane permeability and condensation of nuclear chromatin:
the AO-1 cell type is alive, and is non-apoptotic;
the AO-2 cell type is alive but is also apoptotic;
the AO-3 cell type is dead and non-apoptotic, and
the AO-4 cell type is dead, but is non-apoptotic.

This assay requires microscopic analysis, is labor-intensive, and is skill-intensive while being very sensitive.

Second Assay

An assay for caspase activity is performed in microtiter plates. This caspase activity assay uses a cell-permeable general caspase inhibitor, VAD-FMK (Valylalanylaspartic acid fluoromethyl ketone) bound to a FITC fluorescent tag (FITC is fluorescein isothiocyanate). This inhibitor irreversibly binds to active caspases. This inhibitor has broad specificity for caspases that have been activated by a cleavage reaction event, a cleavage event which only occurs during the process of apoptosis.

Fractionated extracts are evaluated over three orders of magnitude (that is, over a three logarithmic dilution) versus a DMSO vehicle control with appropriate time points as described herein in assays with at least two cell lines, preferably with three cell lines, such as K562 (a leukemia cell line), MCF-7 (a breast cancer cell line) and Caco-2 (a colon cancer cell line) cancer cells. Each of the two cell lines MCF-7 and Caco-2 are adherent. A YO-PRO-1/PI assay is performed on these cell lines, and the activated caspase activity assay is performed on all three of the cell lines.

The use of two different assays for cell death and apoptosis can indicate the mode of death that is occurring in the cancer cell lines. Three categories of chemical agents comprising extracts of ethnopharmacologic plants are discernable. One category comprises extracts that cause necrosis or caspase-independent cell death in cancer cells. These agents display a PI positive result in the first assay, but a negative result in the caspase assay. A second category comprises extracts that cause death by apoptosis alone. These show a YO-PRO-1 positive result in the first assay and a positive result in the caspase assay. A third category comprises extracts that cause death by a combination of apoptotic and non-apoptotic mechanisms. The percentage of cells that die by apoptosis resulting from exposure to this category is less than the total percentage of cells that die. In one aspect, an extract may induce cell death by a different mechanism or signaling pathway in K562 cells than in MCF-7 cells.

In another aspect, an extract of this invention will not induce apoptosis in non-cancerous, immortalized cells but will cause death preferentially in cancer cells.

Primary Screening

In a process of this invention, a plant extract or a composition comprising at least one component that is extracted from a plant is combined with DMSO as a vehicle to form a combination comprising a plant extract, and the combination is exposed to non-tumorigenic and to tumorigenic cells to obtain a comparison between the extract's killing activity and specificity in tumorigenic versus non-tumorigenic cells. Concentrations of the extract or component of the extract are varied over at least three orders of magnitude, for example, 10 μg, 1 μg and 0.1 μg per ml, the concentrations being in micrograms of component of the extract or of the extract per milliliter of the combined volume of component or extract and DMSO. A vehicle control (DMSO) is used for comparison.

A first assay of this invention useful to identify an extract of a plant or a component of an extract of a plant that can kill diseased cells by induction of apoptosis comprises a YO-PRO-1 assay which comprises exposure of the extract-DMSO combination or DMSO control to the cells, which exposure lasts from about 24 to about 48 hours, for example where the cells are MCF-10A breast epithelial cells. In one aspect, a desired extract of this invention exhibits less or no killing activity in non-cancerous cells than in cancer cells. In another aspect, a desired extract of this invention exhibits zero killing activity in non-cancerous cells and from about 1% to 100% killing activity in cancer cells, preferably from about 50% to 100% killing activity in cancer cells, and most preferably 100% killing activity in cancer cells.

This YO-PRO-1 assay indicates death regardless of the pathway. If the extract functions in all types of cells via alternative death pathways, a positive death response is observed. This screening test can be used to identify an extract that exhibits specific or enhanced killing of cancer cells and no killing or less killing of non-cancer cells.

Secondary Screening

A second assay of this invention comprises an annexin V/PI assay which is performed on the same cell lines as used in the first assay of this invention. In this aspect, an extract that exhibits a specific or an enhanced killing of cancer versus non-cancer cells is evaluated in an annexin V/PI assay on the same cells. This assay can confirm the killing or non-killing results from the first screening assay for extracts that exhibit reduced or no killing of non-cancer cells under the same conditions that induce killing of cancer cells. For example, an extract of interest can be identified in this second assay by observing the extract or component of an extract produces lower numbers of dead and/or apoptotic MCF-10A cells than of dead and/or apoptotic MCF-7 cells after treatment with the same concentrations and at the same exposure or incubation times.

Isolation of Components of Ethnobotanical Extracts

Crude plant extracts are fractionated, for example by using a chromatographic method, and the fractions are directly collected on to at least one 96-well microtiter plate, which is then dried and used directly for high throughput screening.

A Hewlett-Packard HPLC unit fitted with an automatic injector and sampler, a diode array-detector (DAD), and a 3D Chem Station is used to separate and to detect components of an ethnobotanical plant extract. The DAD with Chem Station measures UV absorption at several wavelengths in one injection to generate peaks representative of the presence of separated fractions of absorbing components of the extract. The fractions are further purified using preparative HPLC and other chromatographic methods until a pure compound is obtained. The pure compound is analyzed by HPLC-DAD and UV spectra are compared to reduce duplication. The chemical structures of unknown components can be determined with the help of proton and ¹³C nuclear magnetic resonance techniques including COSY and heteronuclear COSY techniques, mass spectroscopy using, for example, low resolution chemical ionization and electron impact mass spectroscopy using a Hewlett-Packard LC-MS system, and ultraviolet/visible/infrared spectroscopy studies as well as by use of X-ray crystallography. High resolution mass spectra and elemental composition will be determined on an A. E. I. MS-902 mass spectrometer. For extract components having relatively high molecular weight above 1000 dalton, fast-atom bombardment or ion spray mass spectrometry can be employed. The presence of chemical functional groups can also be confirmed using well known chemical modification and detection chemistry.

Additionally, extracts and components of extracts can be chemically modified by well known chemical transformation of functional groups, for example by esterification of acids and/or alcohol functional groups or by hydrolysis of ester groups to create new compounds and to facilitate characterization. In the past, we have used all the above methods to identify fungal and plant natural products. Methods useful in chemical transformations and characterizations and used in the following publications are hereby incorporated by reference: Venkatasubbaiah et al. in J. Nat. Products 53: 1628-1630, 1990; in J. Nat. Prod. 54: 1293-1297, 1991; in Phytochemistry, 30: 1471-1474, 1991; in Phytopathology, 81: 243-247, 1991; in Phytopathology, 135: 309-316, 1991; in Mycopathologia, 120: 33-37, 1992; in J. Nat. Prod. 55: 639-643, 1992; in J. Nat. Prod., 55: 461-467, 1992; and in Plant Disease, 79: 1157-1160, 1995.

Each of the extracts or each component of an extract of this invention can be evaluated for its relative efficacy to kill cancer cells versus non-cancer cells, and for its relative efficacy to kill cancer cells by apoptotic versus non-apoptotic killing mechanisms.

Each of the extracts or each component of an extract of this invention can be evaluated for its relative toxicity as a function of concentration and of its cancer cell-specific killing activity in cell models and also in animal models, for example in nude mice models comprising at least one nude mouse inoculated with a plurality of tumorigenic cancer cells comprising at least one of the cell types used in cell screening assays as described herein. All three of the cancer cell lines used in cell culture derived experiments as described herein are tumorigenic in a nude mouse model, and can be used to produce subcutaneous tumors in this model.

Compositions of this invention can be isolated and optionally purified from plant extracts and given as intra-tumoral and/or tail vein injections in nude mice when palpable tumors are formed, and tumors can be measured daily for changes in size. Mice can also be monitored for symptoms related to toxic effects of the compositions of this invention. At selected time points, mice can be sacrificed and mouse tissue samples can be collected from the sacrificed mice for histological analyses. Tissue sections can be stained and analyzed for cell death in tumor and neighboring non-tumor tissue to determine and demonstrate which of the extracts and components of extracts as compositions of this invention have selective cytotoxic effects involving the induction of apoptosis.

A composition of this invention comprising a plant extract can induce apoptosis in at least one cancer cell line and preferably in more than one cancer cell line. For example a composition of this invention comprising a plant extract can induce apoptosis in pancreatic cancer cells, in brain cancer cells, in liver cancer cells, in B cell cancer cells, and in T cell leukemia cancer cells. Extracts and compositions comprising extracts of this invention can be evaluated using a YO-PRO-1 activity assay, and also a caspase activity assay in a primary screening, and then using an Annexin V/PI assay in a secondary screening. MCF-10A cells can be used as a model for non-cancerous breast cells. Compositions of this invention which demonstrate a relatively high killing activity, which activity is identified in a cell based screening assays can be evaluated in a corresponding animal model, for example, comprising a tumor in a nude mice, wherein the tumor comprises cells of the cell line used in the preliminary cell screening assay. The screening of extracts for their ability to induce apoptosis in a cancer cell line can be applied using a broad panel of cancer cell lines to identify compounds and mixtures of compounds which can be effective against one or more cancer cell type or one or more stages of cancer.

Compositions of this invention are useful for induction of apoptosis that leads to cell death in at least one cancer cell line or cancer cell type in the body of patient in need of treatment by an anticancer agent. Compositions of this invention are useful for selective induction of apoptosis in a cancer cell line or cancer cell type in the presence of normal cells in the body of patient in need of treatment by an anticancer agent, wherein the induction of apoptosis leads to cell death in at least one cancer cell line or cancer cell type.

Genetic variations can occur in cancer cells and can involve complex signaling pathways that regulate cell death. The mechanism of induction of apoptosis can differ from one cancer cell line to another and between cancerous and normal cells. For example, many cancer cells have an inactive p53 gene, which can be a critical component in an apoptotic pathway activated by some death signals. These cancer cells are resistant to agents that induce apoptosis via a p53-dependent pathway, but not by a p53-independent pathway. In one aspect of this invention, a composition comprising an extract of a plant can induce apoptosis via a p53-independent pathway. Such a composition is thus a specialized class of agent that is effective against a specific cancer cell type having an inactive p53 gene.

A compound can be cytotoxic to a cancer cell line when administered to cells of the cell line, but it is not necessarily cytotoxic to the cells by a mechanism involving induction of apoptosis in the cancer cells. In addition, apoptosis can proceed by different mechanisms and the mechanism of cell death can differ from one cancer cell line to another. It is an advantage that a compound of this invention can provide unexpected apoptosis-inducing activity leading to cancer cell death in one or more cancer cell lines.

Apoptosis or programmed cell death is a highly organized physiological process to eliminate damaged or abnormal cells. It also plays a major role in embryogenesis where apparently normal cells undergo apoptosis. It is involved in maintaining homeostasis in multicellular organisms. An outstanding feature of apoptosis is it's remarkably stereotyped morphology showing condensation of nuclear heterochromatin, cell shrinkage and loss of positional organization of organelles in the cytoplasm. Although morphological characteristics initially described apoptosis, it is now clear that there is a highly complex molecular process involved. Possible convergence of various events results in the activation of the cellular machinery responsible for apoptosis. The p53 gene that is strongly implicated in animal and human carcinogenesis is a significant regulator of the process of apoptosis. The p53 mutations are now recognized to be the most common genetic changes in human cancers and p53 acts as a tumor suppressor gene. While an apoptotic pathway is related to induction of p53, this pathway is held in check by the antiapoptotic gene bcl-2. The protooncogene bax forms a heterodimer with bcl-2 and accelerates the process of apoptosis. Activation of transcription factor NF-kB involving its translocation to the nucleus has been linked to apoptosis. It can activate both the apoptotic and anti-apoptotic genes.

The nuclear DNA of apoptotic cells shows a characteristic laddering pattern of oligonucleosomal fragments. This results from inter-nucleosomal chromatin cleavage by endogenous endonucleases in multiples of 180 base pairs. This fragmentation is regarded as the hallmark of apoptosis. In cells undergoing apoptosis there is activation of a family of proteases called caspases, so named because they have an obligatory cysteine residue within the active site and cleave peptides adjacent to an aspartic acid residue. Activation of caspases can be directly responsible for many of the molecular and structural changes in apoptosis, which changes include degradation of DNA repair enzyme poly(ADP) ribosepolymerase (PARP) and a dependent protein kinase (DNA-PK), and cleavage of chromatin at inter-nucleosomal sites mediated by caspase-activatedDNase (CAD). Cleavage of cytoskeletal elements and membrane proteins by calpains (calcium binding and thiol-containing proteins) may partly explain the fragmentation of the cells to multiple, spherical ‘apoptotic bodies’. These bodies are typically phagocytosed by adjacent cells or macrophages.

An accepted modality for cancer treatment involves surgery, radiation and drugs, singly or in combination. Cancer chemotherapeutic agents can often provide temporary relief from symptoms, prolongation of life and occasionally, cures. A successful anticancer drug should kill or incapacitate cancer cells without causing excessive damage to normal cells. This ideal situation is achievable by inducing apoptosis in cancer cells. The life span of both normal cells and cancer cells is significantly affected by the rate of apoptosis. Thus, modulating apoptosis can be useful in the management and therapy or prevention of cancer.

To screen plant extracts for apoptotic induction based on the activation of caspase cascades inherent in apoptosis, and to identify components of the extracts that have apoptotic activity, a caspase assay, which detects caspase levels among samples of extracts at varying concentrations, is used.

The recognition site for caspases is marked by three to four amino acids followed by an aspartic acid residue, wherein cleavage occurs after the aspartate. A caspase-3 recognition site comprises the amino acid sequence Asp-Glu-Val-Asp (or DEVD). Additionally, a caspase-7 recognition site comprises the amino acid sequence Asp-Glu-Val-Asp (or DEVD). Caspase-3 and/or caspase-7 can be referred to herein as caspase-3/7. Caspase proteases are present as inactive precursors, wherein inhibitor release or cofactor binding activates the caspase through cleavage at an internal aspartate, for example by autocatalysis or by the action of another protease.

Caspase-3 amplifies the signal from an initiator caspase such as caspase-8 and signifies commitment to cellular disassembly in apoptosis. Caspase-3 cleaves other caspases in the apoptosis mechanism. Caspase-3 also cleaves poly(ADP-ribose) polymerase (PARP), DNA-dependent protein kinase, protein kinase C and actin. Caspase-8 activity obtains relatively early in the cascade of apoptosis. Caspase-8 comprises an initiator of a caspase activation cascade in apoptosis. Caspase-8 is involved in a biological cascade comprising release of cytochrome c from mitochondria, and can activate other caspases such as caspase-3. An amino acid sequence that is recognized by caspase-8 comprises Ile-Glu-Thr-Asp (or IETD).

This invention provides a method of induction of apoptosis in a living cell in a mammal comprising administration to the mammal of a therapeutically effective amount of a pharmaceutical composition comprising a plant extract comprising a compound selected from the group consisting of sclareolide, a sclareolide-like compound, sclareol, a sclareol-like compound, and combinations thereof; and optionally wherein the composition is formulated for administration by an oral, parenteral, transdermal, transmucosal, or subcutaneous route.

This invention provides a method of induction of apoptosis in a living cell in a mammal comprising administration to the mammal of a therapeutically effective amount of a pharmaceutical composition comprising a plant extract comprising a compound selected from the group consisting of sclareolide, a sclareolide-like compound, sclareol, a sclareol-like compound, and combinations thereof, wherein the cell is a benign or malignant tumor cell present in a tissue, organ, fluid, or vessel of a mammal; and optionally wherein the tissue is selected from the group consisting of breast, lung, lymph, prostate, colon and pancreatic.

This invention provides a method of induction of apoptosis in a living cell in a mammal comprising administration to the mammal of a therapeutically effective amount of a pharmaceutical composition comprising a plant extract comprising a compound selected from the group consisting of sclareolide, a sclareolide-like compound, sclareol, a sclareol-like compound, and combinations thereof, wherein the cell is a cancer cell; and optionally wherein the cancer is selected from the group consisting of (breast, lung, lymph, prostate, colon and pancreatic).

This invention provides a method of induction of apoptosis in a living cell in a mammal comprising administration to the mammal of a therapeutically effective amount of a pharmaceutical composition comprising a plant extract comprising a compound selected from the group consisting of sclareolide, a sclareolide-like compound, sclareol, a sclareol-like compound, and combinations thereof, wherein the cell is an abnormal or diseased cell present in a tissue, organ, fluid, or vessel of a mammal.

This invention provides a method of induction of apoptosis in a living cell in a mammal comprising administration to the mammal of a therapeutically effective amount of a pharmaceutical composition comprising a plant extract comprising a compound selected from the group consisting of sclareolide, a sclareolide-like compound, sclareol, a sclareol-like compound, and combinations thereof, wherein the administration is by an oral, parenteral, transdermal, transmucosal, or subcutaneous route.

The following examples are provided in order to further illustrate various embodiments of the invention and are not to be construed as limiting the scope thereof.

### EXAMPLES

### Example 1

Screening for Caspase Activity in Compositions Comprising Plant Extract.

Caspase Assay Protocol

1. Thaw the (100×) substrate Z-DEVD-R110 and Apo-ONE™ Homogeneous Caspase-3/7 Buffer (available from Promega Corporation) to room temperature. Avoid multiple freeze-thaw cycles of the Substrate and Buffer.
2. Mix each component by inversion or vortexing.
3. Dilute the Substrate (1:100) with Buffer to make the desired amount of the Homogeneous Caspase-3/7 Reagent. Store the Reagent, protected from light, at room temperature until use. The Reagent may be stored at 4° C. for 24 hours.
4. Set up assay, blank, and positive or negative control reactions as appropriate.
5. Add Homogeneous Caspase-3/7 Reagent to each well of a black or white 96 well plate, maintaining a 1:1 ratio of Reagent to sample.
6. Gently mix contents by shaking at 300-500 rpm on a plate shaker from 30 seconds up to read time. Incubate the reactions for 30 minutes to 18 hours.
7. Measure the fluorescence of each well at an excitation wavelength of 485±20 nm and an emission wavelength of 530±25 nm.
Data Interpretation: The assay results in fluorescence readings of the individual wells including: a blank control consisting of Homogeneous Caspase-3/7 Reagent+cell culture medium without cells;
negative control consisting of Homogeneous Caspase-3/7 reagent+vehicle-treated cell culture; and
assay consisting of Homogeneous Caspase-3/7 Reagent+Cells with drug addition cell culture samples.

The fluorescence readings are verified with the negative control.

The higher the absorbance or fluorescence emission, the higher the caspase activation, and the higher the therapeutic activity potential.

### Example 2

DNA Fragmentation Assays for Apoptosis Protocol

Protocol I: Triton X-100 Lysis Buffer

In 96 flat-wells plate, incubate 4×10⁶ target cells (40 wells of 10⁵ per well) with desired concentration of effectors (105 target cells per well). After incubation, collect the cell sample in 1.5 ml eppendorf tube, spin down, resuspend with 0.5 ml PBS in 1.5 ml eppendorf tubes, and add 55 ul of lysis buffer for 20 min on ice (4° C.). Centrifuge the eppendorf tubes in cold at 12,000 g for 30 minutes. Transfer the samples to new 1.5 ml eppendorf tubes and then extract the supernatant with 1:1 mixture of phenol:chloroform (gentle agitation for 5 min followed by centrifugation) and precipitate in two equivalence of cold ethanol and one-tenth equivalence of sodium acetate. Spin down, decant, and resuspend the precipitates in 30 ul of deionized water-RNase solution (0.4 ml water+5 ul of RNase) and 5 ul of loading buffer for 30 minutes at 37° C. Also insert 2 ul of Hindi III marker (12 ul of Stock IV) on the outer lanes. Run the 1.2% gel at 5V for 5 min before increasing to 100V.

Protocol II: SDS LysisBuffer

Add SDS lysis buffer to the incubated cell samples (prepared as in Protocol I).
Stock I:Triton X-100 Lysis Buffer 40 ml of 0.5 M EDTA 5 ml of 1 M TrisCl buffer pH 8.0 5 ml of 100% Triton X-100 50 ml of H₂O
Stock II: SDS Lysis Buffer
Stock III: 1.2% Agarose Gel
Prepare a stock of 2 liter of 1×TAE (i.e., 2 liter+40 ml of 5×TAE). Add 2.4 g of agarose power (1.2% agarose) to 200 ml of 1×TAE solution and microwave for 4 min at high power.
Then cool the gel to 50° C. and add 25 ul of ethium bromide before pouring it into the gel plate.
Insert comb and let the gel polymerized.
Stock IV: Hindi III Marker (50 Kb lamda DNA) 4 ul of Hindi III Marker 16 ul of Deionized Water 4 ul of Loading Buffer
   Protocol II: DNA Fragmentation Assay via Dipheylamine

In 24-wells plate, incubate 5×10⁶ targets with desired number of effectors. After incubation, transfer the samples to 15 ml tubes, centrifuge for 30 s at 1500 g, and resuspend in 5 ml of lysis buffer (Stock IV) for 15 min on ice. Centrifuge the samples for 20 min at 27,000 g to separate high-molecular-weight chromatin from cleavage products. Resuspend the pellet in 5 ml of buffer (stock V). Treat the supernatants and pellets with the diphenylamine reagent (Stock VI) and incubate at 370 C for 16-24 hr before colorimetric assessment.
Stock IV: Lysis buffer at pH 8.0 5 mM Tris-HCl 20 mM EDTA 0.5% Triton X-100
Stock V: Buffer at pH 8.0 10 mM Tris-HCl 1 mM EDTA
Stock VI: Diphenylamine reagent (light sensitive) 1.5 g of diphenylamine (steam-distilled) 100 ml acetic acid (redistilled) 1.5 ml of conc. sulfuric acid
On the day of usage, add 0.10 ml of ag acetaldehyde (16 mg/ml) to 20 ml of the diphenylamine reagent.
   Protocol III: DNA Fragmentation via 3H-TdR

5×106 target cells were labeled with 50 μl of 3H-TdR (1 mCi/ml) overnight in 10 ml of media. The next day, the cells were washed 3× with 10 ml of PBS and incubated in 10 ml of media to chase out unincorporated cytoplasmic 3H-TdR. After incubating for 2 hrs, the cells were washed 3× with PBS and then used in lytic assay under the same conditions as the 51 Cr release assay in 96 v-well plates. At the end of the assay, each well was treated with 20 μl of 1.0% Triton-X on ice for 5 minutes, followed by centrifugation at 1500 g in a Beckman T-J6 rotor for 15 minutes. 100 μl of the supernatant were harvested from each well and counted in a scintillation counter. Total count was obtained by resuspending the cells prior to harvesting, and adding 0.1% SDS to solublilize the cells. The % 3H released was calculated with an equation analogous to that for %51 Cr released.)

### Example 3

Acridine Orange/Ethidium Bromide Staining for Apoptosis Cells (AO Staining)

Acridine Orange (AO) is an intercalating fluorescence dye that can enter the nucleus of a cell to stain DNA. This AO-staining method has an advantage of high staining-specificity, but with the disadvantage that samples can only be observed for a short period of time, usually within 24 hours. The AO stain can be used to test cell viabilities in a cell sample in conjunction with propidium iodide (PI). AO/PI fluoresce green under dark field fluorescence microscopy, while nonviable cells fluoresce orange.

Acridine orange (AO)/Ethidium bromide (EtBr) staining for Apoptosis cells (AO staining) Solutions:
(i) AO stock solution: 1 mg/ml as 0.001 g AO+1 ml PBS
(ii) EtBr stock solution: 1 mg/ml: 0.001 g+1 ml PBS
(iii) Working dye solution: 0.1 mg/ml AO stock solution, 0.1 mg/ml EtBr stock solution is prepared by mixing 100 μl of each stock solution plus (+) 800 μl of PBS
Make in gasketed tubes, cover with foil, store at 4° C.
1. Plate out cells in 24-well plate: 3.7×10⁴ cells/well/0.5 ml using the appropriate Isocove's modified dulbecco's medium with 10% calf serum. Cells should be subconfluent.
2. Incubate plate at 37° C. with 10% CO₂ for NMuLi cells or 5% CO₂ for L cells for 24 hours.
3. Check cells and note condition—ex. Subconfluent.
4. Prepare virus inoculums for infection: 5011/well×2 wells/virus/day.
Infection:
5. Aspirate medium from wells, leaving a little (e.g., 10% of medium) behind.
6. Add 50 μl of the virus inoculum to the appropriate wells and 50 μl of gel saline to the mock wells; rock plate to distribute inoculum+“wet” cells.
7. Incubate for 1 hour at 37° C., rocking every 15 minutes to prevent cells from drying out.
8. Add 500 μl/well of medium for a total volume of 550 μl/well.
9. Return to incubator.
10. Check cells at 24 hpi (hours post inoculation) and note their condition, i.e. number (#) of cells, healthy, dying, dead, color of medium.
11. To harvest cells, obtain 13×100 mm glass test tubes and label them to correspond to sample wells.
12. Divide 24-well plate into 3 sections, each section containing 8 wells.
a. Label each section 1 dpi, 2 dpi, and 3 dpi, respectively.
b. Number the 1 dpi wells 1-8 to match the 8 test tubes:
   1 & 2=duplicates
   3 & 4=duplicates
   5 & 6=duplicate
   7 & 8=duplicates
13. Use 1 sterile pasteur pipette for each duplicate pair of wells for each transfer.
14. Transfer the medium from the first 8 wells to the appropriately labeled tubes.
15. Rinse each well with 300 μl PBS/well and transfer to the respective tubes.
16. Trypsinize cells with 300 μl trypsin/well and incubate at 37° C. for 10 minutes; check to see if cells are off the bottom of the wells after the first 5 minutes, and at 10 minutes.
17. Triturate the cells to remove from the well bottoms and transfer to the tubes.
18. Rinse with 300 μl PBS (check wells to make sure cells are all out) and transfer to the tubes.
19. Centrifuge tubes in Sorvall RT6000 for 10 minutes at 1000 rpm, 4° C.
20. Pour off supernatants and touch tube to a paper towel. Will use the backwash to help resuspend the cells.
21. Add 2 μl of the working dye solution (100 μg/ml AO, 100 μg/ml EtBr in PBS at 4° C.) to each tube leaving the pipet tips in the tubes. Final concentration should be 4 ug/ml each of AO & EtBr, 3 to 5×10⁶ cells/ml.
22. Place tubes on ice and cover to protect from light.
23. When ready to view the cells, mix the suspension well and dispense 10 μl onto a slide and place a coverslip on top. View under 20× or 40× objective with FITC filter. Can count 2 samples/slide. View one sample at a time on slide to prevent drying out.
24. Count a total of 100 cells in 4 categories:
Live normal (LN)
Live apoptotic (LA)
Dead normal (DN)
Dead apoptotic (DA)
Live cells fluoresce green and dead cells fluoresce orange.

### Example 4

Cell Cycle Analysis (Flow Cytometry):

To determine the effect of a composition comprising a plant extract of this invention on a cell cycle progression of MOLT3 and H33AJ-JA13 (both from T lineage), cells are incubated with 20 and 10 μg/ml of the composition in DMSO for 4, 8, 24 and 32 hr which is extended to 48 and 56 hr for a concentration of 10 ug/ml. DMSO or 10 μg/ml etoposide are used as controls. At the given times aliquots are removed and the cells are harvested by centrifugation. The cells (1×106 cells) are then resuspended in PBS, washed and resuspended in ice-cold 70% ethanol. DAPI is then added at a final concentration of 1.0 μg/ml. Cells are analyzed for DNA content by quantitation of green fluorescence in a Partec PAS IIIi flow cytometry system (Partec Gmnh, Germany). About 10,000 or more events for H33AJ-JA13 and 16,000 or more for MOLT3 are counted. One-parameter histograms are analyzed using the program for cell cycle analysis supplied from manufacturer.

### Example 5

Information on Apoptosis Induced by Sclareol (PM 16) and Sclareolide (PM 16A)

Following are some of the apoptotic activities induced by these classes of compounds:
1. Significant cytotoxic activity on all cell lines except NAMALWA (Burkitt lymphoma, immature B-cell)
2. Not cytotoxic to resting PBML
3. Cytostatic effect, inhibiting DNA synthesis
4. Effect on DNA synthesis is dose and time dependent
5. Morphological signs consistent with apoptosis on all cell lines tested (MOLT3, H33AJ-JA13; T-cell lines and HL-60; Promyelocytic cell lines). However, DNA cleavage assessment suggests that low molecular weight DNA fragments (DNA laddering) occur in the promyelocytic cell line, HL-60
6. Flow cytometric analysis of the two T-cell lines indicates apoptosis begins at 4 hr of incubation
7. Cell cycle analysis indicates it is phase specific, as a G0/1
8. Appears to kill leukemic cells by activating an apoptotic mechanism and is phase specific.

Having thus generally described the invention, the same will become better understood from the appended claims in which it is set forth in a non-limiting manner.

1. A method of inducing apoptosis in a living cell in a mammal comprising administering to a mammal a therapeutically effective amount of a pharmaceutical composition comprising a plant extract compound selected from the group consisting of sclareolide, a sclareolide-like compound, sclareol, a sclareol-like compound, and combinations thereof.

2. The method of claim 1, wherein the cell is at least one of benign and malignant tumor cell present in a tissue, organ, fluid, or vessel of a mammal.

3. The method of claim 1, wherein the cell is a cancer cell.

4. The method of claim 1, wherein the cell is at least one of abnormal and diseased cell present in at least one of a tissue, organ, fluid, and vessel of a mammal.

5. The method of claim 1, further comprising conducting said administering is by at least one of an oral, parenteral, transdermal, transmucosal, and subcutaneous route.

6. The method of claim 2, further comprising conducting said administering in a manner for inducing apoptosis on cells in tissue from the group consisting of breast, lung, lymph, prostate, colon and pancreatic tissue.

7. The method of claim 3, further comprising conducting said administering in a manner for inducing apoptosis in cancer cells from the group consisting of colon, pancreatic and small lung cells.

8. The method of claim 4, wherein the composition is formulated for conducting said administering by an oral, parenteral, transdermal, transmucosal, or subcutaneous route.

9. The method of claim 1, wherein said plant extract is obtained from plant species selected from the group consisting of:
Acacia farnesiana, Acacia sinuata, Achyranthes aspera, Ageratum conyzoides, Alangium salvifolium, Allium cepa, Amaranthus spinosus, Amorphophallus paeoniifolius, Anthocephalus chinensis, Ardisia solanaceae, Artocarpus integrifolia, Asclepias curasavica, Asparagus racemosus, Atalantia monophylla, Baliospermum montanum, Bauhinia pupurea, Bauhinia tomentosa, Bauhinia variegata, Bidens bipinnata, Bixa orellana, Boerhaavia diffusa, Bombax ceiba, Boswellia serrata, Buchanania lanzan, Bulbostylis barbata, Calotropis gigantea, Capparis zeylanica, Careya arborea, Cassia fistula, Cassia occidentalis, Cassia tora, Cassine glauca, Cedrus deodara, Chomaesyce hirta, Chomaesyce prostrata, Cissampelas pareira, Cissus pallida, Cissus quadrangularis, Clerodendrum serratum, Coccinia indica, Conyza canadensis, Cordia myxa, Coriandrum sativum, Crataeva religiosa, Croton sparsiflorous, Cryptolepis buchanani, Curculigo orchioides, Cyamopsis tetragonoloba, Cyperus rotundus, Datura innoxia, Datura metel, Dolichandrone crispa, Embelia ribes, Erythrina indica, Erythrina stricta, Eupatorium odoratum, Ficus benghalensis, Ficus religiosa, Gardenia latifolia, Glycosmis arborea, Gmelina arborea, Grangea sp., Gymnema sylvestre, Hemidesmus indicus, Heteropogon contortus, Ichnocarpus frutescens, Indoneesiella echiodes, Ipomoea hederifolia, Kalanchoe pinnata, Lannea coromandalica, Leucas aspera, Luffa acutangula, Madhuca indica, Mallotus phillipensis, Melochia corchorifolia, Melothria sp., Mesua nagassarium, Mimosa pudica, Moringa oleifera, Mucuna pruriens, Nerium indicum, Nyctanthes arbor-tristis, Ocimum americanum, Ocimum tenuiflorum, Opuntia monocantha, Oroxylum indicum, Oxalis corniculata, Pandanus fascicularis, Pergularia daemia, Phyllanthus acidus, Physalis minima, Piper longum, Plantago ovata, Polycarpea corymbosa, Polygala erioptera, Polygonum barbatum, Pongamia glabra, Rhus succedanea, Sapindus laurifolius, Sarcostemma acidum, Sida acuta, Smilax zeylanica, Solanum torvum, Solanum trilobatum, Strychnos nux-vomica, Tamarindus indica, Tephrosia purpurea, Tephrosia tinctoria, Terminalia bellirica, Thottea siliquosa, Tinosporia cardifolia, Tragia connabina, Tragia involucrata, Trichopus zeylanicus, Vetiveria zizaniodes, Vitex altissima, Wattakaka volubilis, Xanthium indicum, Ziziphus oenoplia, Amorphophallus paeoniifolius, Cyamopsis tetragonoloba, Coccinia indica, Physalis minima, Calotropis gigentia, Trichopus zeylanicus, Solanum nigrum, Boerhavia diffuse, Indigofera tinctoria, Sida acuta, Anisomeles malabarica, Merremia tridenta, Sida cordifolia, Calotropis procera, Alpinia galangal, Euphorbia hirta and combinations thereof.

10. The method of claim 1, wherein said pharmaceutical composition comprising said compound is prepared for administration in a carrier, wherein said compound comprises from about 0.01% by weight to about 50% by weight of a dosage form of said pharmaceutical composition.

11. The method of claim 1, wherein said compounds have at least one of the chemical structures:

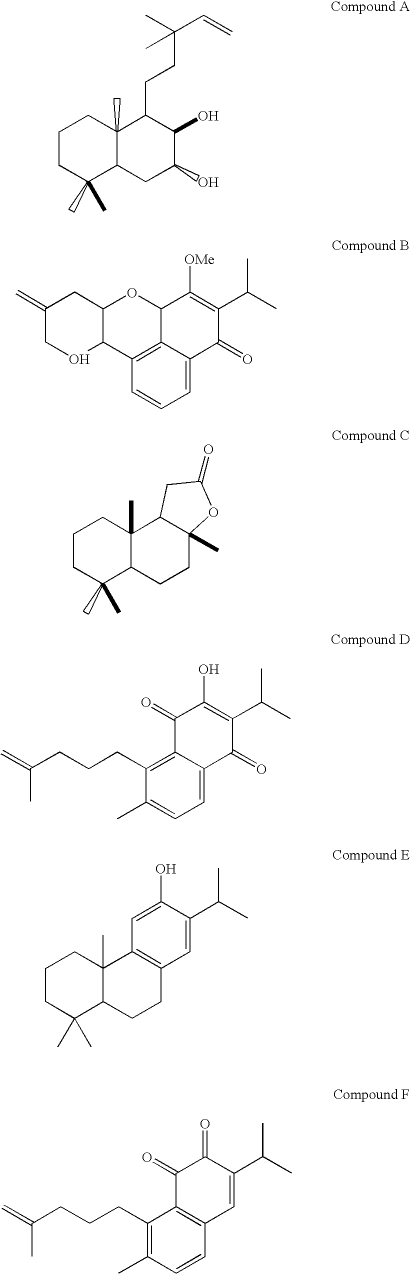

12. A process for the identification of a composition or compound useful in inducing apoptosis in living cells in a mammal, comprising an assay comprising:
a. obtaining an extract of an ethnobotanical plant; and
b. evaluating the activity of the extract in an assay selected from the group consisting of a YO-PRO-1 which exposes cells to an extract carrier combination and measuring killing activity in cancer cells over a period of time, followed by an annexin V/PI assay performed on said YO-PRO-1 cells and measuring killing activity in cancer cells.

13. The process of claim 12, wherein said extract is obtained from plant species selected from the group consisting of:
Acacia farnesiana, Acacia sinuata, Achyranthes aspera, Ageratum conyzoides, Alangium salvifolium, Allium cepa, Amaranthus spinosus, Amorphophallus paeoniifolius, Anthocephalus chinensis, Ardisia solanaceae, Artocarpus integrifolia, Asclepias curasavica, Asparagus racemosus, Atalantia monophylla, Baliospermum montanum, Bauhinia pupurea, Bauhinia tomentosa, Bauhinia variegata, Bidens bipinnata, Bixa orellana, Boerhaavia diffusa, Bombax ceiba, Boswellia serrata, Buchanania lanzan, Bulbostylis barbata, Calotropis gigantea, Capparis zeylanica, Careya arborea, Cassia fistula, Cassia occidentalis, Cassia tora, Cassine glauca, Cedrus deodara, Chomaesyce hirta, Chomaesyce prostrata, Cissampelas pareira, Cissus pallida, Cissus quadrangularis, Clerodendrum serratum, Coccinia indica, Conyza canadensis, Cordia myxa, Coriandrum sativum, Crataeva religiosa, Croton sparsiflorous, Cryptolepis buchanani, Curculigo orchioides, Cyamopsis tetragonoloba, Cyperus rotundus, Datura innoxia, Datura metel, Dolichandrone crispa, Embelia ribes, Erythrina indica, Erythrina stricta, Eupatorium odoratum, Ficus benghalensis, Ficus religiosa, Gardenia latifolia, Glycosmis arborea, Gmelina arborea, Grangea sp., Gymnema sylvestre, Hemidesmus indicus, Heteropogon contortus, Ichnocarpus frutescens, Indoneesiella echiodes, Ipomoea hederifolia, Kalanchoe pinnata, Lannea coromandalica, Leucas aspera, Luffa acutangula, Madhuca indica, Mallotus phillipensis, Melochia corchorifolia, Melothria sp., Mesua nagassarium, Mimosa pudica, Moringa oleifera, Mucuna pruriens, Nerium indicum, Nyctanthes arbor-tristis, Ocimum americanum, Ocimum tenuiflorum, Opuntia monocantha Oroxylum indicum, Oxalis corniculata, Pandanus fascicularis, Pergularia daemia, Phyllanthus acidus, Physalis minima, Piper longum, Plantago ovata, Polycarpea corymbosa, Polygala erioptera, Polygonum barbatum, Pongamia glabra, Rhus succedanea, Sapindus laurifolius, Sarcostemma acidum, Sida acuta, Smilax zeylanica, Solanum torvum, Solanum trilobatum, Strychnos nux-vomica, Tamarindus indica, Tephrosia purpurea, Tephrosia tinctoria, Terminalia bellirica, Thottea siliquosa, Tinosporia cardifolia, Tragia connabina, Tragia involucrata, Trichopus zeylanicus, Vetiveria zizaniodes, Vitex altissima, Wattakaka volubilis, Xanthium indicum, Ziziphus oenoplia, Amorphophallus paeoniifolius, Cyamopsis tetragonoloba, Coccinia indica, Physalis minima, Calotropis gigentia, Trichopus zeylanicus, Solanum nigrum, Boerhavia diffusa, Indigofera tinctoria, Sida acuta, Anisomeles malabarica, Merremia tridenta, Sida cordifolia, Calotropis procera, Alpinia galangal, Euphorbia hirta and combinations thereof.

14. The process of claim 12, wherein said extract is a compound selected from the group consisting of sclareolide, a sclareolide-like compound, sclareol, a sclareol-like compound, and combinations thereof.

15. The process of claim 12, wherein said extract comprises a compound having at least one of the chemical structure:

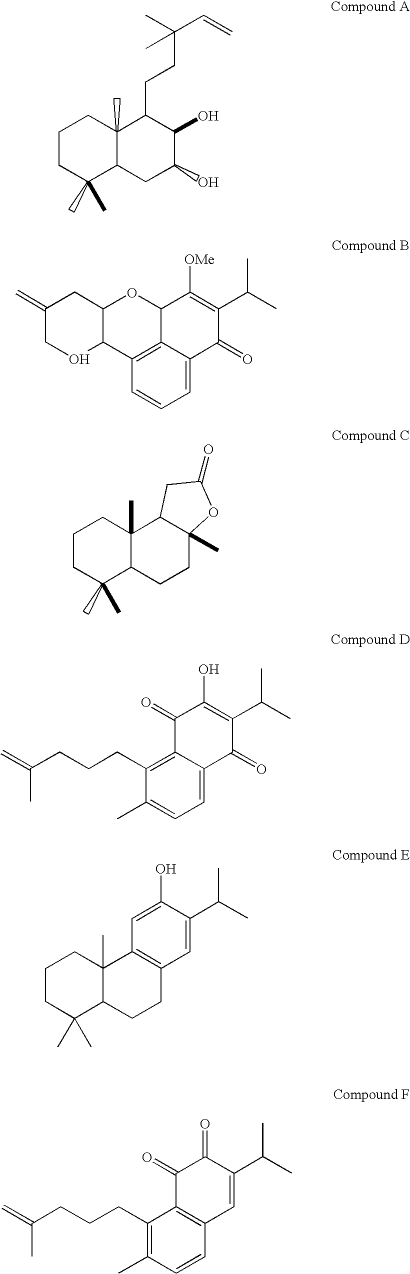

16. A process for the identification of a composition or compound useful in inducing apoptosis in a living cells in a mammal, comprising an assay comprising:
a. obtaining an extract of an ethnobotanical plant, said extract being a compound selected from the group consisting of sclareolide, a sclareolide-like compound, sclareol, a sclareol-like compound, and combinations thereof; and
b. evaluating the activity of the extract in inducing apoptosis in an assay selected from the group consisting of, detection and quantification of caspase activity, YO-PRO-1/Propidicin iodide staining, Amerexin V/Propidicum iodide flow cytometry, and Acridine orange/Ethidium bromide (AO/EtBr) staining.

17. The process of claim 16, wherein said extract is obtained from plant species extract is obtained from plant species selected from the group consisting of:
Acacia farnesiana, Acacia sinuata, Achyranthes aspera, Ageratum conyzoides, Alangium salvifolium, Allium cepa, Amaranthus spinosus Amorphophallus paeoniifolius, Anthocephalus chinensis, Ardisia solanaceae, Artocarpus integrifolia, Asclepias curasavica, Asparagus racemosus, Atalantia monophylla, Baliospermum montanum, Bauhinia pupurea, Bauhinia tomentosa, Bauhinia variegata, Bidens bipinnata, Bixa orellana, Boerhaavia diffusa, Bombax ceiba, Boswellia serrata, Buchanania lanzan, Bulbostylis barbata, Calotropis gigantea, Capparis zeylanica, Careya arborea, Cassia fistula, Cassia occidentalis, Cassia tora, Cassine glauca, Cedrus deodara, Chomaesyce hirta, Chomaesyce prostrata, Cissampelas pareira, Cissus pallida, Cissus quadrangularis, Clerodendrum serratum, Coccinia indica, Conyza canadensis, Cordia myxa, Coriandrum sativum, Crataeva religiosa, Croton sparsiflorous, Cryptolepis buchanani, Curculigo orchioides, Cyamopsis tetragonoloba, Cyperus rotundus, Datura innoxia, Datura metel, Dolichandrone crispa, Embelia ribes, Erythrina indica, Erythrina stricta, Eupatorium odoratum, Ficus benghalensis, Ficus religiosa, Gardenia latifolia, Glycosmis arborea, Gmelina arborea, Grangea sp., Gymnema sylvestre, Hemidesmus indicus, Heteropogon contortus, Ichnocarpus frutescens, Indoneesiella echiodes, Ipomoea hederifolia, Kalanchoe pinnata, Lannea coromandalica, Leucas aspera, Luffa acutangula, Madhuca indica, Mallotus phillipensis, Melochia corchorifolia, Melothria sp., Mesua nagassarium, Mimosa pudica, Moringa oleifera, Mucuna pruriens, Nerium indicum, Nyctanthes arbor-tristis, Ocimum americanum, Ocimum tenuiflorum, Opuntia monocantha, Oroxylum indicum, Oxalis corniculata, Pandanus fascicularis, Pergularia daemia, Phyllanthus acidus, Physalis minima, Piper longum, Plantago ovata, Polycarpea corymbosa, Polygala erioptera, Polygonum barbatum, Pongamia glabra, Rhus succedanea, Sapindus laurifolius, Sarcostemma acidum, Sida acuta, Smilax zeylanica, Solanum torvum, Solanum trilobatum, Strychnos nux-vomica, Tamarindus indica, Tephrosia purpurea, Tephrosia tinctoria, Terminalia bellirica, Thottea siliquosa, Tinosporia cardifolia, Tragia connabina, Tragia involucrata, Trichopus zeylanicus, Vetiveria zizaniodes, Vitex altissima, Wattakaka volubilis, Xanthium indicum, Ziziphus oenoplia, Amorphophallus paeoniifolius, Cyamopsis tetragonoloba, Coccinia indica, Physalis minima, Calotropis gigentia, Trichopus zeylanicus, Solanum nigrum, Boerhavia diffusa, Indigofera tinctoria, Sida acuta, Anisomeles malabarica, Merremia tridenta, Sida cordifolia, Calotropis procera, Alpinia galangal, Euphorbia hirta and combinations thereof
18. The process of claim 15, wherein said extract comprises a compound having at least one of the chemical structure: [chemical structure]
